SEQUENCE LISTING (2 sequences)

SEQ ID NO: 1 - protein, 9 aa, Artificial Sequence
  Synthetic
HSEGTFTSD

SEQ ID NO: 2 - DNA, 27 bp, Artificial Sequence
  Synthetic
cactcagagggaacgtttaccagtgac

(19) United States
(12) Patent Application Publication
(10) Pub. No.: US 2005/0222036 A1
(43) Pub. Date: Oct. 6, 2005

(54) Peptide compositions with effects on blood glucose

(75) Inventors: Matthew J. During, Philadelphia, PA (US); Colin N. Haile, Katy, TX (US); Lei Cao, Haddonfield, NJ (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: THOMAS JEFFERSON UNIVERSITY, Philadelphia, PA (US)

(21) Appl. No.: 10/405,089

(22) Filed: Apr. 1, 2003

Related U.S. Application Data
(63) Continuation-in-part of application No. 09/939,472, filed on Aug. 24, 2001, now abandoned.
(60) Provisional application No. 60/227,631, filed on Aug. 24, 2000.
(60) Provisional application No. 60/369,249, filed on Apr. 1, 2002.

(51) Int. Cl. A61K 38/08

(52) U.S. Cl. 514/14; 514/15

(57) ABSTRACT

The present invention provides compositions and methods for ameliorating neurological or memory disorders and improving learning and cognition through the increase of cyclic AMP. Gilatides, peptides comprising the nine amino acid sequence (SEQ ID NO:1), and functional analogs thereof are disclosed to modulate neurological activity when administered to a subject. The methods of the invention can be used to prevent or treat neurological disorders as well as improve memory retention and acquisition. In addition, the invention can be used to modulate insulin levels and blood glucose. The invention includes pharmaceutical compositions comprising a therapeutically or prophylactically effective amount of a Gilatide peptide or a functional analog thereof.

### PRIORITY

The present invention is a continuation-in-part of U.S. application Ser. No. 09/939,472 filed Aug. 24, 2001, which claims priority to U.S. Provisional Application No. 60/227,631 filed Aug. 24, 2000, entitled “Novel Peptide with Effects on Cerebral Health.” In addition, the present invention claims priority to U.S. Provisional Application No. 60/369,249 filed Apr. 1, 2002, entitled “Novel Peptide with Effects on Cerebral Health.”

### FIELD OF THE INVENTION

The present invention relates to the field of neurology, and in particular, the construction and use of peptides and their derivatives with cognitive enhancing and/or neuroprotective activity.

### BACKGROUND OF THE INVENTION

Dementia, a structurally-caused, permanent or progressive decline of intellectual function, is one of the most serious disorders facing the elderly population. Dementia, which normally results in a loss of short-term and/or long-term memory, interferes substantially with social as well as economic activities. Memory loss is characteristic of the normal aging process as well as of many neurological disorders. Shockingly, approximately 80% of people over 30 complain of some degree of memory loss. The risk of dementia is correlated with age and doubles every five years after the age of 60. Studies report that up to 50% of people over the age of 85 are afflicted with this disorder. An estimated 60-80% of elderly nursing home residents are affected by this disease. Currently, treatment of dementia in the elderly focuses primarily on environmental issues rather than biochemical causes.

Notably, the various forms of dementia are attributable to different causes. Many neurological disorders, such as Alzheimer's disease, can lead to forms of dementia. For example, Alzheimer-type dementia has been attributed to specific cellular and histological degenerative processes resulting in brain atrophy and the loss of cells from the basal forebrain, cerebral cortex, and other brain areas. Stroke, head trauma, and epilepsy can also lead to memory impairment. Epilepsy, a brain disorder in which neurons signal abnormally, can cause strange sensations, emotions, and behavior, or sometimes convulsions, muscle spasms, and loss of consciousness.

Existing medications for neurological disorders and memory weaknesses are not always well tolerated, nor have they been proven effective in alleviating symptoms of dementia and memory loss. In addition, drugs, such as anti-epileptic drugs, can interfere with the effectiveness of other medications, such as oral contraceptives. Furthermore, while gingko biloba, piracetam, and various other “smart drugs” are being actively marketed, no proven memory-enhancing drug exists.

With the increasing lifespan of people, the lack of drugs that treat the biochemical causes of neurological disorders and memory impairment is becoming an acute problem. Thus, there exists a need in the art for drugs that can alleviate dementia and improve cognition and memory.

### SUMMARY OF THE INVENTION

The present invention provides compositions and methods for ameliorating neurological or memory disorders and improving learning and cognition through the increase of cyclic AMP. Gilatides, peptides comprising the nine amino acid sequence (SEQ ID NO:1), and functional analogs thereof are disclosed to modulate neurological activity when administered to a subject. The methods of the invention can be used to prevent or treat neurological disorders as well as improve memory retention and acquisition. The invention includes pharmaceutical compositions comprising a therapeutically or prophylactically effective amount of a Gilatide peptide or a functional analog thereof.

The present invention is based, in part, on the discovery of the remarkable cognitive and neuroprotective effects of the nine amino acid sequence HSEGTFTSD (SEQ. ID. NO: 1), named by the inventors as “Gilatide”. Such Gilatide peptides are homologous, but not necessarily identical, to fragments of both GLP-1 (amino acids 7-15) as well as Exendin-4 (amino acids 1-9), a peptide isolated from the saliva of the Gila Monster. Where these native proteins have a glycine in position 2, the Gilatide peptide of the present invention preferably has a serine in this position. The substitution of serine for glycine in position 2 increases the stability of the synthetic peptide in comparison to that of both GLP-1 and Exendin-4.

In one aspect of the invention, small Gilatide peptides and analogs can be synthesized that induce cAMP production. In one embodiment, the Gilatide peptide or functional analog thereof comprises less than 20 amino acids. In one embodiment, the Gilatide peptide or functional analog thereof comprises less than 15 amino acids. In another embodiment, the Gilatide peptide or functional analog thereof comprises less than 10 amino acids.

In one embodiment, the present invention comprises a nucleic acid comprising a sequence that encodes a polypeptide with the amino acid sequence of SEQ ID NO: 1, such that upon administration to a subject, the polypeptide increases cAMP. In another embodiment, the nucleic acid sequence comprises SEQ ID NO:2 or a degenerate variant of SEQ ID NO: 2 encoding a polypeptide having at least nine amino acids whereby the polypeptide increases cAMP. In yet another embodiment, the present invention comprises an expression vector comprising the nucleic acid sequence that encodes a polypeptide with the amino acid sequence of SEQ ID NO: 1 operably linked to an expression control sequence. In one embodiment, the expression vector is an adenovirus vector. In another embodiment, the invention comprises a cultured cell comprising an expression vector encoding a polypeptide with the amino acid sequence of SEQ ID NO: 1. In yet another embodiment, the cultured cell transfected with the expression vector, or a progeny of the cell, expresses the Gilatide polypeptide or analog thereof. In yet another aspect, the present invention discloses a method of producing a protein comprising culturing the cell under conditions permitting expression under the control of the expression control sequence. In another embodiment, the invention discloses a purified peptide, the amino acid sequence comprising HSEGTFTSD (SEQ ID NO:1) or analog thereof.

In one embodiment, the present invention discloses a pharmaceutical composition comprising a pharmaceutically acceptable carrier and a therapeutically effective amount of the purified Gilatide peptide or analog thereof, the amino acid sequence of which comprising HSEGTFTSD (SEQ ID NO:1) or said sequence with conservative amino acid substitutions. In another embodiment, administration of the therapeutically effective amount of purified peptide is intranasal. In yet another embodiment, administration of the therapeutically effective amount of purified peptide is intraperitoneal.

In one aspect, the present invention provides methods and compositions to regenerate neural tissue that has been damaged by a CNS (central nervous system) or neurological disorder. In one embodiment, the present invention can be used to prevent, ameliorate, slow the progression, or delay onset of a neurological disorder. In one embodiment, the neurological disorders include, but are not limited to, head injury, spinal cord injury, seizure, stroke, epilepsy and ischemia. Such neurological disorders include neurodegenerative disorders. Neurological disorders include neurodegenerative diseases such as, but not limited to, epilepsy, Huntington disease, Parkinson's disease, attention deficit disorder (ADD), neuropsychiatric syndromes, ALS, and Alzheimer's disease. Further neurological disorders include CNS damage resulting from infectious diseases such as viral encephalitis, bacterial or viral meningitis and CNS damage from tumors. In another aspect, administering a therapeutically effective amount of a Gilatide peptide or functional analog is neuroprotective. In addition, the present invention may also find use in enhancing the cell-based therapies used to repair damaged spinal cord tissue following a spinal cord injury, or used to treat or prevent various demyelinating and dysmyelinating disorders, such as Pelizaeus-Merzbacher disease, multiple sclerosis, various leukodystrophies, post-traumatic demyelination, and cerebrovascular accidents.

In a preferred embodiment, the methods and compositions of the present invention can be used to treat or reduce Alzheimer's disease. Alzheimer's disease (AD) is a degenerative brain disease, the incidence of which rapidly increases with advancing age. Certain populations of brain cells progressively die. Recently modem imaging techniques have revealed how the medial temporal lobe area, which contains the hippocampus (a vital structure for learning and memory generally in humans and for certain types of spatial learning in animals) progressively shrinks as Alzheimer's disease progresses.

The method of administering a therapeutically effective amount of a Gilatide peptide or functional analog can be selected from the group comprising intraperitoneal, intracerebroventricular, intradermal, intramuscular, intravenous, subcutaneous, and intranasal. In a preferred embodiment, a therapeutically effective amount of a Gilatide peptide or functional analog is delivered by intranasal administration.

In another aspect, the present invention discloses a method for modulating a memory disorder in a subject, comprising administering to the subject a therapeutically effective amount of a Gilatide peptide or functional analog thereof, such that the administration of the Gilatide peptide or functional analog produces an amelioration of the memory disorder. In one embodiment, the method further comprises administering a therapeutically effective amount of a Gilatide peptide or functional analog thereof prior to onset of the memory disorder. In another embodiment, the administration of a therapeutically effective amount of a Gilatide peptide or functional analog thereof decreases memory acquisition time. In yet another embodiment, the administration of a therapeutically effective amount of a Gilatide peptide or functional analog thereof increases memory retention time. In as yet another aspect, the invention discloses a method for preventing or delaying the onset of a memory disorder in a subject, the method comprising administering to the subject a prophalactically effective amount of Gilatide or analog thereof, in a pharmaceutically acceptable carrier.

In one aspect, the compositions and methods of the present invention can be used to reduce memory disorders. A memory disorder refers to a diminished level of mental registration, retention or recall of past experiences, knowledge, ideas, sensations, thoughts or impressions. Memory disorders may affect short and long-term information retention, facility with spatial relationships, memory (rehearsal) strategies, and verbal retrieval and production. In another aspect, the compositions and methods of the present invention can be used to enhance memory performance including, but not limited to, improving or increasing the mental faculty by which to register, retain or recall past experiences, knowledge, ideas, sensations, thoughts, or impressions.

This invention supports and encompasses the use of Gilatide peptides and analogs as potent and long lasting cognitive-enhancing drugs. The effect of Gilatide is evident 24 hours after administration of the peptide and is still present one week after a single administration. The primary effect appears to be on acquisition of memory and not consolidation. Moreover, Gilatide is devoid of behavioral activating or anti-nociceptive effects and, thus, appears to be specific for memory enhancement.

In as yet another aspect, the present invention provides a method for modulating cyclic AMP in a subject, comprising administering to the subject a therapeutically effective amount of a Gilatide peptide or functional analog thereof that modulates cAMP, such that the administration of the Gilatide peptide or functional analog modulates cAMP levels in the subject. In a preferred embodiment, the administration of the Gilatide peptide or functional analog thereof increases cAMP in the subject. In another embodiment, the administration of the Gilatide peptide or functional analog thereof increases CREB (cAMP Responsive Element Binding Protein).

In yet another aspect, the present invention provides a method of modulating the MAP kinase pathway in a subject comprising administering to the subject a therapeutically effective amount of a Gilatide peptide or functional analog thereof that modulates at least one enzyme in the MAP kinase pathway, such that the modulation produces an amelioration in the progression of the memory disorder. In a preferred embodiment, the administration of the Gilatide peptide or functional analog thereof increases MAP kinase in the subject.

In one embodiment, intracerebroventricular glucagon-like peptide 1 (GLP-1), a gut peptide that is expressed in the brain along with its receptor, and the 9mer HSEGTFTSD (SEQ ID NO: 1) are disclosed to enhance associative and spatial learning.

In another embodiment, SEQ ID NO:1 strongly enhances associative and spatial learning via GLP-1 receptors (GLP-1R) linked to an ERK/MAP kinase signal transduction pathway. In yet another embodiment, peptides comprising SEQ ID NO: 1 or active analogs thereof are active when administered peripherally. In yet another aspect, GLP-1R and analogs thereof can be used to enhance cognition. In another embodiment, GLP-1R and analogs thereof can be used as a neuroprotective agent.

In one aspect of the invention, glucagon-like peptide 1 receptor (GLP-1R) in the brain is a target for cognitive-enhancing agents. In another aspect, GLP-1R is a target for neuroprotective agents. In one embodiment, Gilatide and functional analogs interact with GLP-1R to modulate cAMP levels. In one embodiment, Gilatide and functional analogs interact with GLP-1R to modulate CREB (cAMP Responsive Element Binding Protein) expression, secretion or activity. In another embodiment, Gilatide and functional analogs thereof interact with GLP-1R to modulate the MAP kinase pathway. In another embodiment, Gilatide and functional analogs thereof interact with GLP-1R to modulate insulin production or secretion. GLP-1R deficient mice have a phenotype characterized by a learning deficit. In contrast, rats over-expressing GLP-1R in the hippocampus display markedly enhanced spatial and contextual learning. GLP-1R deficient mice also have enhanced seizure severity and neuronal injury following kainate administration, whereas systemic administration of a peptide comprising SEQ ID NO:1 in wild-type animals prevents kainate-induced apoptosis of hippocampal neurons.

In another aspect of the invention, it has been discovered that Gilatide or functional analogs increase cyclic AMP. In one embodiment, Gilatide or functional analogs thereof increase CREB signaling in the brain. It previously has been demonstrated that drugs that facilitate CREB are neuroprotective. Thus, Gilatide, in addition to its neurotropic activity (i.e., cognitive facilitation) can be neuroprotective. The base peptide described herein, Gilatide, represents an example of a peptide that can be used to treat, either prophylactically or therapeutically, nervous system or neurological disorders associated with neuronal loss or dysfunction and facilitate learning, memory and cognition. The scope of this invention is not limited to this example; the example is used to illustrate the technology of the present invention. Those skilled in the art are familiar with peptide synthesis techniques so that any analog, derivative, fragment, or mimetic that retains the biological activity of Gilatide in cellular or animal models can be used for the purposes of the present invention.

In another aspect, the present invention provides a method for modulating blood glucose in a subject by administering to the subject a therapeutically effective amount of a Gilatide peptide or functional analog thereof that modulates insulin secretion, such that the administration of the Gilatide peptide or functional analog produces an increase in insulin, thereby modulating blood glucose levels. In one embodiment, the therapeutically effective amount of Gilatide peptide of functional analog can be administered intraperitoneally. In another method according to the invention, Gilatide peptides can be used to modulate a glucose-metabolism disorder in a subject. Such glucose-metabolism disorders can include, but are not limited to, the group consisting of obesity, diabetes, anorexia nervosa, insulin resistance, glucose intolerance, hyerinsulinemia, Syndrome X, hypercholesterolemia, hyperlipoproteinemia, hypertriglyceridemia, atherosclerosis, and diabetic renal disease.

### BRIEF DESCRIPTION OF THE FIGURES

FIG. 1 is a bar graph of an ELISA of media from RINm5f cells for insulin confirming bioactivity of synthesized Gilatide peptide; Vehicle (▪), GLP-1 (□), GLP-1+Exendin (9-39) ( [symbol] ), Gilatide (slashed box), and Gilatide+Exendin (9-39) (double slashed box);

FIG. 2 is a bar graph showing an increased mean latency to move into the dark compartment of a passive avoidance apparatus in which they had experienced an adverse stimulus of rats pretreated with 10 μg Gilatide versus control (Vehicle (VEH) treated) at various time points following the initial adverse stimulus;

FIG. 3A is a bar graph showing that various doses of intracerebroventricular (i.c.v.) GLP-1 and Gilatide ([Ser(2)]exendin(1-9)) enhanced latency in associative learning (passive avoidance), similar to vasopressin;

FIG. 3B is a bar graph showing that co-infusion of exendin (9-39) blocks the effects of GLP-1 and Gilatide ([Ser(2)]exendin(1-9)) but not vasopressin;

FIGS. 4A is a graph showing no difference in acquisition between Gilatide treated and control groups based upon the results of a Morris Water Maze (MWM) task assay in which latency to find a submerged platform was measured;

FIG. 4B is a graph showing that 10 μg, 30 μg, and 60 μg Gilatide facilitates retention for 48 hours of spatial learning in the Morris Water Maze task assay;

FIG. 5A is a graph of the distance traveled to find a hidden platform in the MWM following administration GLP-1, Gilatide ([Ser(2)]exendin(1-9)) or control (Vehicle);

FIG. 5B is a graph demonstrating that both peptides GLP-1 and Gilatide decreased swimming speed compared to vehicle (P<0.05);

FIG. 6 depicts the representative swimming path tracings of five individual rats on day 5 in the MWM;

FIG. 7 is a bar graph showing mean (±S.E.M.) latencies (acquisition) to move into the dark compartment from a bright compartment of a passive avoidance apparatus of rats pretreated via various routes of administration of Gilatide or Vehicle (VEH) +P=1.0; *P=<0.05, (t-test) vs. VEH;

FIG. 8 is a bar graph of mean (±S.E.M.) latencies (retention) to move into the dark compartment from a bright compartment of a passive avoidance apparatus latency rats pretreated with various levels of Gilatide, Vehicle (VEH), or Nicotine, +P=0.1; *P<0.05, (t-test) vs. VEH, **P<0.05 vs. Nicotine;

FIG. 9A shows the enhancement of learning and memory by intranasal [Ser(2)]exendin(1-9) where [Ser(2)]exendin(1-9) (up-slashed, 3, 10, and 30 μg), but not GLP-1 (□) enhanced latency in PA comparable to vasopressin (down-slashed, 0.3 μg; +P=0.01 for [Ser(2)]exendin(1-9) 3 μg; *P<0.05 for [Ser(2)]exendin(1-9) 10 μg and vasopressin);

FIG. 9B shows that co-treatment with exendin (9-39) blocked the effects of [Ser(2)]exendin(1-9) (up slashed) but not vasopressin (down-slashed) (*P<0.05) resulting in effects similar in Exendin (9-39) only (dotted);

FIG. 10A is a graph showing that intranasal treatments of GLP-1, Gilatide, and Arecoline did not affect acquisition of spatial learning compared to the control;

FIG. 10B is a graph showing that [Ser(2)]exendin(1-9) (up slashed, 30 μg) enhanced retention of spatial learning, comparable to arecoline (wavy lined, 0.3 mg s.c.; **P<0.01), over that of vehicle (▪) or GLP-1 (□);

FIG. 11 is a graph showing the effects of [Ser(2)]exendin(1-9) (up slashed, 10 μg), arecoline (wavy, 0.3 mg) and vasopressin (down-slashed, 0.3 μg) on repeated testing in PA in which [Ser(2)]exendin(1-9) enhanced retention to a greater degree than arecoline (wavy) and vasopressin (down slashed) (*P<0.05);

FIG. 12A is a bar graph illustrating that acute administration of Gilatide has no significant effect on food intake of rats following 18 hours of deprivation;

FIG. 12B is a bar graph illustrating that acute administration of Gilatide has no significant effect on water intake of rats following 18 hours of deprivation;

FIG. 13 is a bar graph showing the effects of Gilatide on consolidation of learning for rats treated intranasally with 10 μg/kg Gilatide 20 minutes (TRN-TXT, grey) or 24 hours (TXT-DYL-TRN, black) after the conditioning session;

FIG. 14 is a bar graph of latency, measured in a passive avoidance apparatus, for rats pretreated with various levels of Gilatide with or without an Exendin-4 antagonist, or vehicle (VEH) illustrating that co-treatment with the Exendin-4 antagonist (9-39) (10 μg) completely blocked enhancement of associative learning by Gilatide (10 μg) (*P=0.03 vs. Gilatide 10 μg, combination vs. VEH, ##P=0.43) and increasing the dose of Gilatide (20 μg) surmounted the antagonism (vs. VEH, **P=0.04);

FIG. 15 is a bar graph of mean latencies measured in a passive avoidance apparatus for rats pretreated with Gilatide, saline, scrambled peptide, or vehicle (VEH);

FIG. 16 is a bar graph of % control latency versus dose of intranasal administration of Gilatide ([Ser(2)]exendin(1-9)) in GLP-1R+/+ (▪) and GLP-1R−/− (□) mice demonstrating that Gilatide enhanced latency times in GLP-1R+/+ (*P<0.05) but not in GLP-1R−/− mice in the PA paradigm;

FIG. 17 is a bar graph of % freezing behavior demonstrating contextual fear conditioning in which GLP-1R−/− (□) (**P<0.01) showed significant decrements in contextual fear conditioning compared to GLP-1R+/+ mice (▪);

FIG. 18A is a graph demonstrating that Gilatide ([Ser(2)]exendin(1-9)) (1 μg, slashed circles) produced a trend towards a decrease in latency compared to vehicle-treated mice (▪) in acquisition of spatial learning in wild type mice (F=2.72(1,72); P=0.10);

FIG. 18B is a bar graph demonstrating that Gilatide ([Ser(2)]exendin(1-9)) at doses of 1 μg (slashed box) and 3 μg ( [symbol] ) decreased latency in the retention of spatial learning in GLP-1R +/+ mice (*P<0.05) compared to vehicle (▪);

FIG. 19A is a graph demonstrating that Gilatide ([Ser(2)]exendin(1-9)) (slashed circles) did not enhance acquisition in GLP-1R−/− mice compared to vehicle (▪);

FIG. 19B is a bar graph demonstrating no difference in latency to find a visual platform for wild-type (▪) and GLP-1R−/− mice (□);

FIG. 20 is a bar graph demonstrating that that Gilatide ([Ser(2)]exendin(1-9)) at doses of 1 μg (slashed box) and 3 μg ( [symbol] ) did not enhance retention of spatial learning in GLP-1R−/− mice compared to vehicle (▪);

FIG. 21 is a graph showing the decreased distance traveled to locate the hidden platform in rats with over-expression of GLP-1R in hippocampus (rAAV) (◯) compared to EGFP controls (▪);

FIG. 22 is a bar graph demonstrating that GLP-1R overexpression (□), and arecoline (*P<0.05) ( [symbol] ) enhanced freezing behavior in contextual fear conditioning compared to naïve (▪) and EFGP ( [symbol] ) controls;

FIG. 23 is a bar graph showing no effect of Gilatide on locomotor activity of rats where mean (±S.E.M.) distance traveled (cm) was measured over 30 minutes in rats administered VEH (5% β cyclodextrin) or Gilatide (10-60 μg, intranasal, in 5% β cyclodextrin);

FIG. 24 is a bar graph illustrating the effects of Gilatide on nociception based upon the results of a tail immersion assay where mean (±S.E.M.) tail flick latencies following pretreatment with VEH (5% β cyclodextrin) or Gilatide (10-60 μg, intranasal, in 5% β cyclodextrin) was measured;

FIG. 25A is a graph showing that intranasal Gilatide (slashed) enhanced MAP kinase immunoreactivity in the cytosolic fraction of the hippocampus of rats compated to vehicle (▪), *P=0.05;

FIG. 25B is a graph showing that intranasal Gilatide (slashed) enhanced MAP kinase immunoreactivity in the nuclear fraction of the hippocampus of rats compated to vehicle (▪); *P=0.05;

FIG. 26 is a bar graph demonstrating that the effects of intranasal Gilatide on associative learning in rats was blocked by administration of a specific MEK inhibitor, PD98059 (5 μg, i.c.v.), post-training (slashed), but not pre-training (□);

FIG. 27 is a bar graph showing that the latency to seizure onset in response to 20 mg/kg kainic acid (KA) was significantly lower (*P<0.05) in GLP-1−/− (□) compared to wild-type GLP-1+/+ mice (▪); and

FIG. 28 is a bar graph showing that the maximum seizure severity score was greater in GLP-1−/− mice (□) compared to wild-type GLP-1+/+ mice (▪).

### DETAILED DESCRIPTION

The present invention concerns the construction and use of peptides and their derivatives with cognitive enhancing and/or neuroprotective activity. The practice of the present invention employs methods of molecular biology, neurology, and peptide synthesis.

So that the invention may more readily be understood, certain terms are defined:

The term “peptide,” as used herein, is used in reference to a functional or active analog, of Gilatide or a Gilatide-derived peptide. Peptide means a compound containing naturally occurring amino acids, non-naturally occurring amino acids or chemically modified amino acids, provided that the compound retains the bioactivity of Gilatide.

As used herein, the term “amino acid” refers to one of the twenty naturally occurring amino acids, including, unless stated otherwise, L-amino acids and D-amino acids. The term amino acid also refers to compounds such as chemically modified amino acids including amino acid analogs, naturally occurring amino acids that are not usually incorporated into peptides such as norleucine, and chemically synthesized compounds having properties known in the art to be characteristic of an amino acid, provided that the compound can be substituted within a peptide such that it retains its biological activity. For example, glutamine can be an amino acid analog of asparagine, provided that it can be substituted within an active fragment, derivative or analog of Gilatide that retains its bioactivity or function in cellular and animal models. Other examples of amino acids and amino acids analogs are listed in Gross and Meienhofer, The Peptides: Analysis, Synthesis, Biology, Academic Press, Inc., New York (1983), which is incorporated herein by reference. An amino acid also can be an amino acid mimetic, which is a structure that exhibits substantially the same spatial arrangement or functional groups as an amino acid but does not necessarily have both the α-amino and α-carboxyl groups characteristics of an amino acid.

The terms “functional” or “bioactive,” as used interchangeably herein, mean a Gilatide-derived peptide having a non-amino acid chemical structure that mimics the structure of Gilatide or a Gilatide-derived peptide and retains the bioactivity and function of Gilatide in cellular and animal models. The function may include an improved desired activity or a decreased undesirable activity. Such a mimetic generally is characterized as exhibiting similar physical characteristics such as size, charge or hydrophobicity in the same spatial arrangement found in Gilatide or the Gilatide-derived peptide counterpart. A specific example of a peptide mimetic is a compound in which the amide bond between one or more of the amino acids is replaced, for example, by a carbon-carbon bond or other bond well known in the art (see, for example, Sawyer, Peptide Based Drug Design, ACS, Washington (1995), which is incorporated herein by reference). Non-limiting tests for a-functional Gilatide are disclosed below. The peptides of the present invention are intended to be functional in at least one bioactivity assay. Specifically, when the peptide is subject to in vivo and/or in vitro testing conditions, a modification results. Tests for functionality are described in the Examples section of the specification. For example, an increase in cAMP, an increase in memory, an increase in CREB (cAMP responsive element binding protein) expression, production, or secretion, and/or an increase in neuroprotection can result following the addition of the peptide.

The terms “neurological disorder” or “CNS disorder,” as used interchangeably herein, refer to an impairment or absence of a normal neurological function or presence of an abnormal neurological function in a subject. For example, neurological disorders can be the result of disease, injury, and/or aging. As used herein, neurological disorder also includes neurodegeneration which causes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times.

Neurological disorders include, but are not limited to, head injury, spinal cord injury, seizures, stroke, dementia, memory loss, attention deficit disorder (ADD), epilepsy, and ischemia. Neurological disorders also include neurodegenerative diseases. Neurodegeneration can occur in any area of the brain of a subject and is seen with many disorders including, but not limited to, Amyotrophic Lateral Sclerosis (ALS), multiple sclerosis, Huntington's disease, Parkinson's disease and Alzheimer's disease.

Further neurological disorders include CNS (central nervous system) damage resulting from infectious diseases such as viral encephalitis, bacterial or viral meningitis and CNS damage from tumors. The neuroprotective and/or neural regenerative strategy of the present invention can be also be used to improve the cell-based replacement therapies used to treat or prevent various demyelinating and dysmyelinating disorders, such as Pelizaeus-Merzbacher disease, multiple sclerosis, various leukodystrophies, post-traumatic demyelination, and cerebrovasuclar accidents. Disorders of the central nervous system further include mental disorders such as mood disorders (i.e., depression, bipolar disorder), anxiety disorders, memory disorders and schizophrenic disorders. In addition, the present invention may also find use in enhancing the cell-based therapies used to repair damaged spinal cord tissue following a spinal cord injury.

The term “glucose-metabolism disorder” as used herein, is intended to refer to any disorder relating to glucose uptake or release, as well as, insulin expression, production, secretion, or usage. The glucose-metabolism disorder can be selected from, but not limited to, the group consisting of obesity, diabetes, anorexia nervosa, insulin resistance, glucose intolerance, hyerinsulinemia, Syndrome X, hypercholesterolemia, hyperlipoproteinemia, hypertriglyceridemia, atherosclerosis, and diabetic renal disease.

The term “memory disorder,” as used herein, refers to a diminished mental registration, retention or recall of past experiences, knowledge, ideas, sensations, thoughts or impressions. Memory disorder may affect short and/or long-term information retention, facility with spatial relationships, memory (rehearsal) strategies, and verbal retrieval and production. The term memory disorder is intended to include dementia, slow learning and the inability to concentrate. Common causes of a memory disorder are age, severe head trauma, brain anoxia or ischemia, alcoholic-nutritional diseases, drug intoxications, and neurodegenerative diseases. For example, a memory disorder is a common feature of neurodegenerative diseases, such as Alzheimer's disease (i.e. Alzheimer-type dementia). Memory disorders also occur with other kinds of dementia such as AIDS Dementia; Wernicke-Korsakoff's related dementia (alcohol induced dementia); age related dementia, multi-infarct dementia, a senile dementia caused by cerebrovascular deficiency, and the Lewy-body variant of Alzheimer's disease with or without association with Parkinson's disease. Creutzfeldt-Jakob disease, a spongiform encephalopathy caused by the prion protein, is a rare dementia with which memory disorder is associated. Loss of memory is also a common feature of brain-damaged patients. Non-limiting examples of causes of brain damage which may result in a memory disorder include stroke, seizure, an anaesthetic accident, ischemia, anoxia, hypoxia, cerebral edema, arteriosclerosis, hematoma or epilepsy; spinal cord cell loss; and peripheral neuropathy, head trauma, hypoglycemia, carbon monoxide poisoning, lithium intoxication, vitamin (B1, thiamine and B12) deficiency, or excessive alcohol use. Korsakoff's amnesic psychosis is a rare disorder characterized by profound memory loss and confabulation, whereby the patient invents stories to conceal his or her memory loss. It is frequently associated with excessive alcohol intake. Memory disorder may furthermore be age-associated; the ability to recall information such as names, places and words seems to decrease with increasing age. Transient memory loss may also occur in patients, suffering from a major depressive disorder, after electro-convulsive therapy.

The terms “pharmacological agent” as used herein, refers to the compound, or compounds, of the present invention that are used to modulate the neuronal activity in a subject. Preferably, the neuronal modulating pharmacological agent of the present invention is a peptide comprising the Gilatide sequence SEQ ID NO:1. By way of non-limiting example, a neurological modulating pharmacological agent according to the present invention is a peptide comprising SEQ ID NO: 1, peptide comprising SEQ ID NO:1 with conservative amino acid substitutions, peptide comprising SEQ ID NO: 1 with non-amino acid substitutions, and active analogs of peptides comprising SEQ ID NO:1. The peptides comprising SEQ ID NO:1 are intended to be functional in at least one bioactivity assay. Specifically, when the peptide is subject to in vivo and/or in vitro testing conditions, a modification results. Tests for functionality are described in the Examples section of the specification. For example, an increase in cAMP, an increase in memory, an increase in CREB (cAMP responsive element binding protein) expression, production, or secretion, and/or an increase in neuroprotection can result from the addition of the peptide.

The term “therapeutically effective amount” refers to an amount effective, at dosages and for periods of time necessary, to achieve the desired therapeutic result. A therapeutically effective amount of Gilatide may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The term “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “subject” as used herein refers to any living organism capable of eliciting an immune response. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The terms “Gilatide,” “Gilatide peptide,” and “[Ser(2)]exendin(1-9),” as used interchangeably herein, refer to a neuroprotective factor of any origin, which is substantially homologous and functionally equivalent to peptides comprising SEQ ID NO:1. (HSEGTFTSD) or peptides comprising SEQ ID NO: 1 with conservative amino acid or non-amino acid substitutions. Gilatides may exist as monomers, dimers or other multimers in their biologically active form. Thus, the term “Gilatide” as used herein encompasses active monomeric Gilatides, as well as active multimeric Gilatides, active glycosylated and non-glycosylated forms of Gilatide, active truncated forms of the molecule, and active larger peptides comprising SEQ ID NO:1. The term Gilatide is intended to include peptides comprising SEQ ID NO:1 that have been post-translationally modified. By “functionally equivalent” as used herein, is meant a Gilatide peptide that retains some or all of the neuroprotective properties, but not necessarily to the same degree, as a native Gilatide molecule. Gilatides, comprising the nine amino acids SEQ ID NO:1, can be less than 50 amino acids in length. Gilatides, comprising the nine amino acids SEQ ID NO:1, can be less than 40 amino acids in length, preferably less than 30 amino acids in length, more preferably less than 25 amino acids in length. More preferably, Gilatides, comprising the nine amino acids SEQ ID NO:1, can be less than 20 amino acids in length. Most preferably, Gilatides, comprising the nine amino acids SEQ ID NO:1, can be less than 15 amino acids in length, but not less than 9 amino acids. Gilatides, comprising the nine amino acids SEQ ID NO:1, can be less than 10 amino acids in length. Methods for making polynucleotides encoding for Gilatides are known in the art and are described further below.

“Homology” refers to the percent similarity between two polynucleotide or two polypeptide moieties. Two polynucleotide, or two polypeptide sequences are “substantially homologous” to each other when the sequences exhibit at least about 50%, preferably at least about 75%, more preferably at least about 80%-85%, preferably at least about 90%, and most preferably at least about 95%-99% or more sequence similarity or sequence identity over a defined length of the molecules. As used herein, substantially homologous also refers to sequences showing complete identity to the specified polynucleotide or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis of similarity and identity, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence similarity and identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent similarity of a particular nucleotide sequence to a reference sequence can be determined using the homology algorithm of Smith and Waterman with a default scoring table and a gap penalty of six nucleotide positions.

Another method of establishing percent similarity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence similarity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The term “Gilatide analog” as used herein, refers to a biologically active derivative of the a Gilatide peptide, or a fragment of such a derivative, that retains desired activity, such as neuroprotective activity in the assays described herein. In general, the term “analog” as used herein, is intended to mean functional derivatives or fragments that is related structurally and functionally to another substance. An analog contains a modified structure from the parent substance, in this case Gilatide, and maintains the function of the parent substance, in this instance, the biological function or activity of Gilatide in cellular and animal models. The biological activity of the analog may include an improved desired activity or a decreased undesirable activity. The analog need not, but can be synthesized from the other substance. For example, a Gilatide analog can be a compound structurally related to Gilatide, but not necessarily made from Gilatide. Analogs of the instant invention, include, but are not limited to, analogs of the synthetic peptide, Gilatide, that are homologous to glucagon, Exendin-4 and glucagon-like peptides. In general, the term “analog” refers to compounds having a native polypeptide sequence and structure with one or more amino acid additions, substitutions (generally conservative in nature) and/or deletions, relative to the native molecule, so long as the modifications do not destroy neuroprotective activity. Preferably, the analog has at least the same neuroprotective activity as the native molecule. The term analog is intended to include peptides comprising the SEQ ID NO:1 (HSEGTFTSD) with one or more amino acid substitutions (preferably conservative) as well as peptides comprising the SEQ ID NO:1 (HSEGTFTSD) with amino acid or non-amino acid substitutions to the sequence. Gilatide analogs, comprising the nine amino acids SEQ ID NO:1, can be less than 50 amino acids in length. Gilatide analogs, comprising the nine amino acids SEQ ID NO:1, can be less than 40 amino acids in length, preferably less than 30 amino acids in length, more preferably less than 25 amino acids in length. More preferably, Gilatide analogs, comprising the nine amino acids SEQ ID NO:1, can be less than 20 amino acids in length. Most preferably, Gilatide analogs, comprising the nine amino acids SEQ ID NO:1, can be less than 15 amino acids in length. Gilatide analogs, comprising the nine amino acids SEQ ID NO:1, can be less than 10 amino acids in length. Methods for making polynucleotides encoding for Gilatide analogs are known in the art and are described further below.

For Gilatide addition analogs, amino acid sequence additions typically include N- and/or C-terminal fusions ranging in length from one residue to polypeptides containing a hundred or more residues, as well as internal additions of single or multiple amino acid residues. Internal additions generally range from about 1-10 residues, more typically from about 1-5 residues, and usually from about 1-3 amino acid residues, or any integer within the stated ranges. Examples of N-terminal addition variants include the fusion of a heterologous N-terminal signal sequence to the N-terminus of Gilatide as well as fusions of amino acid sequences derived from the sequence of other neuroprotective factors.

Gilatide substitution analogs have at least one amino acid residue of SEQ ID NO: 1 removed and a different residue inserted in its place. Such substitution variants include allelic variants, which are characterized by naturally occurring nucleotide sequence changes in the species population that may or may not result in an amino acid change. Particularly preferred substitutions are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity.

For example, the Gilatide molecule may include up to about 8 conservative or non-conservative amino acid substitutions, or preferably up to about 3 conservative or non-conservative amino acid substitutions, so long as the desired function of the molecule remains intact. One having ordinary skill in the art may readily determine regions of the molecule of interest that can tolerate change using techniques well known in the art.

The term “Gilatide analog” means an active Gilatide polypeptide as defined above or below having at least about 50% amino acid sequence identity with a full-length native sequence PRO polypeptide sequence as disclosed herein, or any other fragment of a full-length Gilatide polypeptide sequence as disclosed herein. Such Gilatide polypeptide variants include, for instance, Gilatide polypeptides wherein one or more amino acid residues are added, or deleted, at the N- or C-terminus of the full-length native amino acid sequence. Ordinarily, a Gilatide polypeptide variant will have at least about 50% amino acid sequence identity, alternatively at least about 55% amino acid sequence identity, alternatively at least about 60% amino acid sequence identity, alternatively at least about 65% amino acid sequence identity, alternatively at least about 70% amino acid sequence identity, alternatively at least about 75% amino acid sequence identity, alternatively at least about 80% amino acid sequence identity, alternatively at least about 85% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a full-length native sequence Gilatide polypeptide sequence as disclosed herein, or any other specifically defined fragment of a full-length Gilatide polypeptide sequence as disclosed herein. Ordinarily, Gilatide analog polypeptides are at least about 9 amino acids in length, alternatively at least about 10 amino acids in length, alternatively at least about 15 amino acids in length, alternatively at least about 20 amino acids in length, alternatively at least about 25 amino acids in length, alternatively at least about 30 amino acids in length, alternatively at least about 35 amino acids in length, alternatively at least about 40 amino acids in length, alternatively at least about 45 amino acids in length, alternatively at least about 50 amino acids in length, alternatively at least about 55 amino acids in length, alternatively at least about 60 amino acids in length, alternatively at least about 65 amino acids in length, or more.

The term “percent (%) amino acid sequence identity” with respect to the Gilatide polypeptide sequences identified herein is defined as the percentage of amino acid residues in a candidate sequence that are identical with the amino acid residues in the specific Gilatide polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

Percent amino acid sequence identity values may be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing (a) the number of matching identical amino acid residues between the amino acid sequence of the Gilatide analog of interest having a sequence derived from the native Gilatide peptide and the comparison amino acid sequence of interest (i.e., the sequence against which the Gilatide analog of interest is being compared which may be a Gilatide variant polypeptide) as determined by WU-BLAST-2 by (b) the total number of amino acid residues of the Gilatide analog of interest. For example, in the statement “a polypeptide comprising the amino acid sequence A which has or having at least 80% amino acid sequence identity to the amino acid sequence B”, the amino acid sequence A is the comparison amino acid sequence of interest and the amino acid sequence B is the amino acid sequence of the Gilatide analog of interest.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y, where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The terms “Gilatide variant polynucleotide” or “Gilatide variant nucleic acid sequence” means a nucleic acid molecule which encodes an active Gilatide polypeptide as defined below and which has at least about 50% nucleic acid sequence identity with a nucleotide acid sequence encoding a full-length sequence Gilatide polypeptide sequence as disclosed herein (SEQ ID NO: 2), or any other fragment of a full-length Gilatide polypeptide sequence as disclosed herein. The Gilatide nucleic acid sequence (SEQ ID NO: 2) comprises:
cac tca gag gga acg ttt acc agt gac

Ordinarily, a Gilatide variant polynucleotide will have at least about 50% nucleic acid sequence identity, alternatively at least about 55% nucleic acid sequence identity, alternatively at least about 60% nucleic acid sequence identity, alternatively at least about 65% nucleic acid sequence identity, alternatively at least about 70% nucleic acid sequence identity, alternatively at least about 75% nucleic acid sequence identity, alternatively at least about 80% nucleic acid sequence identity, alternatively at least about 85% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity with a nucleic acid sequence encoding a full-length sequence Gilatide polypeptide sequence as disclosed herein, or any other fragment of a full-length Gilatide polypeptide sequence as disclosed herein.

Ordinarily, Gilatide variant polynucleotides are at least about 27 nucleotides in length, alternatively at least about 30 nucleotides in length, alternatively at least about 60 nucleotides in length, alternatively at least about 90 nucleotides in length, alternatively at least about 120 nucleotides in length, alternatively at least about 150 nucleotides in length, alternatively at least about 180 nucleotides in length, alternatively at least about 210 nucleotides in length, alternatively at least about 240 nucleotides in length, alternatively at least about 270 nucleotides in length, alternatively at least about 300 nucleotides in length, alternatively at least about 600 nucleotides in length, alternatively at least about 900 nucleotides in length, or more.

“Percent (%) nucleic acid sequence identity” with respect to Gilatide-encoding nucleic acid sequences identified herein is defined as the percentage of nucleotides in a candidate sequence that are identical with the nucleotides in the Gilatide nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software.

By “vector” is meant any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

By an “AAV vector” is meant a vector derived from an adeno-associated virus serotype, including without limitation, AAV-1, AAV-2, AAV-3, AAV-4, AAV-5, AAV-6, AAV-7 and AAV-8. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

“AAV helper functions” refer to AAV-derived coding sequences which can be expressed to provide AAV gene products that, in turn, function in trans for productive AAV replication. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors.

The term “AAV helper construct” refers generally to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for lytic AAV replication; however, helper constructs lack AAV ITRs and can neither replicate nor package themselves. AAV helper constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945. A number of other vectors have been described which encode Rep and/or Cap expression products. See, e.g., U.S. Pat. Nos. 5,139,941 and 6,376,237.

The term “accessory functions” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

The term “accessory function vector” refers generally to a nucleic acid molecule that includes nucleotide sequences providing accessory functions. An accessory function vector can be transfected into a suitable host cell, wherein the vector is then capable of supporting AAV virion production in the host cell. Expressly excluded from the term are infectious viral particles as they exist in nature, such as adenovirus, herpesvirus or vaccinia virus particles. Thus, accessory function vectors can be in the form of a plasmid, phage, transposon, or cosmid.

In particular, it has been demonstrated that the full-complement of adenovirus genes are not required for accessory helper functions. In particular, adenovirus mutants incapable of DNA replication and late gene synthesis have been shown to be permissive for AAV replication. Ito et al., (1970) J. Gen. Virol. 9:243; Ishibashi et al, (1971) Virology 45:317. Similarly, mutants within the E2B and E3 regions have been shown to support AAV replication, indicating that the E2B and E3 regions are probably not involved in providing accessory functions. Carter et al., (1983) Virology 126:505. However, adenoviruses defective in the E1 region, or having a deleted E4 region, are unable to support AAV replication. Thus, E1A and E4 regions are likely required for AAV replication, either directly or indirectly. Laughlin et al., (1982) J. Virol. 41:868; Janik et al., (1981) Proc. Natl. Acad. Sci. USA 78:1925; Carter et al., (1983) Virology 126:505. Other characterized Ad mutants include: E1B (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al.(1983), supra; Carter (1995)). Although studies of the accessory functions provided by adenoviruses having mutations in the E1B coding region have produced conflicting results, Samulski et al., (1988) J. Virol. 62:206-210, recently reported that E1B55k is required for AAV virion production, while E1B19k is not. In addition, International Publication WO 97/17458 and Matshushita et al., (1998) Gene Therapy 5:938-945, describe accessory function vectors encoding various Ad genes.

Particularly preferred accessory function vectors comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such vectors are described in International Publication No. WO 01/83797.

By “recombinant virus” is meant a virus that has been genetically altered, e.g., by the addition or insertion of a heterologous nucleic acid construct into the particle.

By “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, e.g., “sense” or “antisense” strands, can be packaged into any one AAV virion and both strands are equally infectious.

A “recombinant AAV virion,” or “rAAV virion” is defined herein as an infectious, replication-defective virus including an AAV protein shell, encapsidating a heterologous nucleotide sequence of interest which is flanked on both sides by AAV ITRs. A rAAV virion is produced in a suitable host cell which has had an AAV vector, AAV helper functions and accessory functions introduced therein. In this manner, the host cell is rendered capable of encoding AAV polypeptides that are required for packaging the AAV vector (containing a recombinant nucleotide sequence of interest) into infectious recombinant virion particles for subsequent gene delivery.

The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties, such as a nucleotide integration vector and other nucleic acid molecules, into suitable host cells.

The term “host cell” denotes, for example, microorganisms, yeast cells, insect cells, and mammalian cells, that can be, or have been, used as recipients of an AAV helper construct, an AAV vector plasmid, an accessory function vector, or other transfer DNA. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “nucleic acid” sequence refers to a DNA or RNA sequence. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil-, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

“Operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

By “purified” or “isolated” when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

The term “pharmaceutically acceptable carrier” as used herein, refers to a carrier that is conventionally used in the art to facilitate the storage, administration, and/or the biological activity of a regulatory agent. A carrier may also reduce any undesirable side effects of the regulatory agent. A suitable carrier should be stable, i.e., incapable of reacting with other ingredients in the formulation. It should not produce significant local or systemic adverse effect in recipients at the dosages and concentrations employed for treatment. Such carriers are generally known in the art. Suitable carriers for this invention include those conventionally used for large stable macromolecules such as albumin, gelatin, collagen, polysaccharide, monosaccharides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), and the like. Water, saline, aqueous dextrose, and glycols are preferred liquid carriers, particularly (when isotonic) for solutions. The carrier can be selected from various oils, including those of petroleum, animal, vegetable or synthetic origin, for example, peanut oil, soybean oil, mineral oil, sesame oil, and the like. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. The compositions can be subjected to conventional pharmaceutical expedients, such as sterilization, and can contain conventional pharmaceutical additives, such as preservatives, stabilizing agents, wetting, or emulsifying agents, salts for adjusting osmotic pressure, buffers, and the like. Other acceptable components in the pharmaceutical composition include, but are not limited to, isotonicity-modifying agents such as water, saline, and buffers including phosphate, citrate, succinate, acetic acid, and other organic acids or their salts. Typically, the pharmaceutically acceptable carrier also includes one or more stabilizers, reducing agents, anti-oxidants and/or anti-oxidant chelating agents. The use of buffers, stabilizers, reducing agents, anti-oxidants and chelating agents in the preparation of protein-based compositions, particularly pharmaceutical compositions, is well known in the art. See, Wang et al. (1980) J. Parent. Drug Assn. 34(6):452-462; Wang et al. (1988) J. Parent. Sci. Tech. 42:S4-S26 (Supplement); Lachman et al. (1968) Drug and Cosmetic Industry 102(1):36-38, 40, and 146-148; Akers (1988) J. Parent. Sci. Tech. 36(5):222-228; and Methods in Enzymology, Vol. XXV, ed. Colowick and Kaplan, “Reduction of Disulfide Bonds in Proteins with Dithiothreitol,” by Konigsberg, pp. 185-188.

Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. See Wang (1980) supra at page 455. Suitable salts and isotonicifiers include sodium chloride, dextrose, mannitol, sucrose, trehalose, or the like. Where the carrier is a liquid, it is preferred that the carrier is hypotonic or isotonic with oral, conjunctival, or dermal fluids and has a pH within the range of 4.5-8.5. Where the carrier is in powdered form, it is preferred that the carrier is also within an acceptable non-toxic pH range.

Suitable reducing agents, which maintain the reduction of reduced cysteines, include dithiothreitol (DTT also known as Cleland's reagent) or dithioerythritol at 0.01% to 0.1% wt/wt; acetylcysteine or cysteine at 0.1% to 0.5% (pH 2-3); and thioglycerol at 0.1% to 0.5% (pH 3.5 to 7.0) and glutathione. See Akers (1988) supra at pages 225-226. Suitable antioxidants include sodium bisulfite, sodium sulfite, sodium metabisulfite, sodium thiosulfate, sodium formaldehyde sulfoxylate, and ascorbic acid. See Akers (1988) supra at page 225. Suitable chelating agents, which chelate trace metals to prevent the trace metal catalyzed oxidation of reduced cysteines, include citrate, tartarate, ethylenediaminetetraacetic acid (EDTA) in its disodium, tetrasodium, and calcium disodium salts, and diethylenetriamine pentaacetic acid (DTPA). See, e.g., Wang (1980) supra at pages 457-458 and 460-461, and Akers (1988) supra at pages 224-227.

I. Gilatide Sequence

The present invention is based, in part, on the discovery of the sequence homology of the glucagon-like peptides (GLP) and GLP family members. The glucagon-like peptides (GLP) are a family of peptides that, prior to this invention, were not associated with central effects on the brain and nervous system. Analysis of GLP and GLP family members has identified a homologous domain of these proteins with therapeutic activity. BLAST analysis revealed highly conserved residues within the GLP super family in both vertebrates and invertebrates (See Table 1). These peptides include glucagon itself, and the GLP-1R agonist, exendin-4, which is extracted from the saliva of the Gila monster lizard, Heloderma suspectum. In contrast, exendin(9-39), a N-terminus truncated GLP-1/Exendin 4 acts as a GLP-1R antagonist (J. P. Raufman et al. J. Biol. Chem. 266, 2897 (1991)). Heloderma suspectum exendin 4, Heloderma suspectum proglucagon (LPII), and Heloderma suspectum proglucagon (LPI) can be found in GenBank Accession Nos: U77613, U77612, and U77611, respectively. The therapeutic methods and compositions of the present invention encompass use of the full-length GLP-1 and the Gilatide sequences, the 9mer SEQ ID NO: 1, HSEGTFTSD, and peptides comprising SEQ ID NO:1 as well as active analogs thereof. Where these native proteins have a glycine in position 2, the Gilatide peptide of the present invention has a serine in this position. The substitution of serine for glycine in position 2 increases the stability of the synthetic peptide in comparison to that of both GLP-1 and Exendin-4. Of interest, the glucagon protein sequence of both the torpedo and the common dogfish also have a serine in the position 2.

TABLE 1

Amino acid sequences of GLP superfamily; PACAP-38, pituitary
adenylate cyclase-activating peptide, VIP, vasoactive

intestinal polypeptide.

| | |
|---|---|
| Glucagon | HSQGTFTSDYSKYLDSRRAQDDFVQWLMNT |
| GLP-1 (7-36) | HAEGTFTSDVSSVLEGQAAKEFIAWLVKGR |
| GLP-2 | HADGSFSDEMNTILDNLAARDFINWLIQTKITD |
| Exendin-4 | HGEGTFTSDLSKQMEEEAVRLFIEWLKNGGPSSGAPPPS |
| Exendin (9-39) | DLSKQMEEEAVRLFIEWLKNGGPSSGAPPPS |
| PACAP-38 | HSDGIFTDSYSRYRKQMAVKKYLAAVLGKRYKQRVKNK |
| VIP | HSDAVFTDNYTRLRKQMAVKKYLNSILN |
| Gilatide | HSEGTFTSD |
| (SEQ ID NO:1) | |
| [Ser(2)]exendin(1-9) | |

The present invention is based in part on the identification of this small domain (<10 amino acids) and the verification that peptides comprising these 9 amino acids retain cAMP activation ability. A small peptide that retains the essential bioactivity is preferable since it is more stable and would be able to more easily pass through the blood-brain barrier (BBB). In this invention, Gilatide peptides and analogs are shown to have cognitive-enhancing efficacy following peripheral administration (See Examples).

II. Memory Enhancement

In one aspect, the present invention provides methods and compositions for modulating memory disorders. Thus, the compositions and methods of the present invention can be used to prevent, delay onset, or treat memory disorders. The present invention can increase mental registration, retention or recall of past experiences, knowledge, ideas, sensations, thoughts or impressions. In a preferred embodiment, the present invention increases short and/or long-term information retention, facility with spatial relationships, memory (rehearsal) strategies, and verbal retrieval and production. Gilatide and analogs have been shown to increase both passive learning and spatial learning (See Examples 3, 4, 7, and 8).

In another aspect, Gilatide and analogs can increase cyclic AMP. Gilatide peptides and analogs can also increase CREB (cAMP responsive element binding protein) production, secretion and/or activity. In addition, Gilatide peptides and analogs can increase the phosphorylation of CREB. Example 12 demonstrates that intranasal administration of Gilatide and analogs can increase CREB expression in the cytosolic and nuclear fraction of the hippocampus of rats.

Learning and memory in animals, both vertebrates and invertebrates, involves what is commonly termed synaptic plasticity, i.e., a mechanism by which a given input is associated with enhanced or facilitated output. The most commonly established physiological model of such learning is long term potentiation (LTP), by which repeated excitatory pulses, i.e., titanic stimuli, lead to a long lasting potentiation of the stimulated synapse. The molecular mechanism of this synaptic potentiation and plasticity is starting to be unraveled, with the data suggesting a change in gene expression mediated via transcriptional activation. The transcription factors with the most convincing and supportive data are members of the cyclic AMP (cAMP) responsive element binding protein (CREB) family. Loss of plasticity and impaired learning and memory have been demonstrated in studies involving the delivery of mutant CREB in model systems as well as studies of CREB knockout mice. Conversely, activating CREB or overexpressing CREB has been shown to induce a super-learning phenotype. Furthermore, cAMP response element binding protein (CREB) has been shown to be essential in the conversion of short- to long-term memory (Fox, K. Neuroscience, 2002, 111(4): 799-814; Zhang et al. Neuroscience 2003 117(3) 707-713; Scott R et al. J Mol Neurosci 2002 19(1-2):171-177). In summary, cAMP regulated CREB, and CREB may regulate the expression of the transcription factor, Zif268, whose expression is triggered by LTP and learning (See, for example, Silva A J. J Neurobiol 2003 54(1):224-237).

There are a large number of endogenous peptides that have effects on learning and memory in mammalian model systems. These include vasoactive intestinal protein (VIP), vasopressin or anti-diuretic hormone (ADH), and corticotrophin releasing hormone (CRH). Each of these native peptides, however, retains pleiotropic actions, including influences on neuroendocrine function, as well as potential anxiogenic or arousal effects that are likely to limit any potential applications. Moreover, these peptides generally are only effective if directly delivered into the central nervous system (CNS).

In yet another aspect, the present invention can modulate glutamate. In one embodiment, Gilatide peptides and analogs can modulate glutamate directly. In another embodiment, Gilatide peptides and analogs can modulate glutamate through interaction with its receptors. Glutamate, a predominant excitatory neurotransmitter in the CNS, is predicted to play an important role in cognition, memory, neuronal plasticity, learning, and some neurological disorders such as epilepsy, stroke and neurodegeneration (Schoepp et al. Trends Pharmacol Sci. 1993 14(1):13-20). Two distinct classes of receptors, ionotropic and metabotropic receptors, regulate the function of glutamate. Ionotropic receptors (iGluRs) are glutamate-activated ion channels which mediate “fast” excitatory actions of glutamate. Metabotropic glutamate receptors (mGluRs) are part of the 7-transmembrane domain G-protein-coupled receptor family. Eight functional subtypes of metabotropic glutamate receptors have been identified (Coutinho V. et al. Neuroscientist. 2002, 8(6):551-61), and have a range of physiological effects, including increasing the membrane excitability of neurons by inhibiting Ca²⁺ dependent K⁺ conductances, inhibiting and potentiating excitatory transmission supported by ionotropic glutamate receptors, and inhibiting the afterhyperpolarization that follows bursts of actions potentials in the dentate gyrus and CA1 neurons in the hippocampus. These receptors are also involved in long-term potentiation.

In as yet another aspect, Gilatide peptides and analogs increase MAP kinase production, secretion, and/or activity in the brain. In a preferred embodiment, the increase of MAP kinase activity is in the hippocampus. In another embodiment, Gilatide peptides and analogs activate the extracellular signal-regulated protein kinase (ERK)/mitogen-activated protein kinase (MAP) kinase pathway with nuclear translocation of p42 MAP kinase, which is associated with long-term memory. Example 12 demonstrates that intranasal administration of Gilatide increases MAP kinase expression in both the cytosolic and nuclear fractions of rat hippocampus. Furthermore, Example 12 shows that the increase in associative learning following adminstration of intranasal Gilatide is lost if a specific MEK inhibitor is given immediately after training.

Activation of the mitogen-activated protein kinase (MAPK) cascade has been shown to play an important role in cognition in multiple species, including humans, as well as synaptic plasticity in the CA1 area of rat hippocampus (Weeber et al. Neuron, 2002, 33: 845). Hippocampal MAPK activation is regulated by both the protein kinase A (PKA) and protein kinase C (PKC) systems. A variety of neuromodulatory neurotransmitter receptors (i.e., metabotropic glutamate receptors, muscarinic acetylcholine receptors, dopamine receptors, and beta-adrenergic receptors) couple to MAPK activation via these two cascades. PKC is a powerful regulator of CREB phosphorylation in area CA1. Since MAPK activation is necessary for increased CREB phosphorylation in response to the activation of this kinase, MAPK plays a critical role in transcriptional regulation via PKC. Studies indicate a diversity in the regulation of MAPK in the hippocampus suggesting that the MAPK cascade may play a broad role in regulating gene expression in long-term forms of hippocampal synaptic plasticity (Roberson et al. J. Neurosci. 1999, 19(11): 4337-4348.)

The MAPK family consists of key regulatory proteins that are known to regulate cellular responses to both proliferative and stress signals. MAPKs consist of several enzymes, including a subfamily of extracellular signal-activated kinases (ERK1 and ERK2) and stress-activated MAPKs. There are three distinct groups of MAPKs in mammalian cells: a) extracellular signal-regulated kinases (ERKs), b) c-Jun N-terminal kinases (JNKs) and c) stress activated protein kinases (SAPKs).

PKC activation or other factors (e.g. increases in free intracellular Ca²⁺) activates small proteins called Ras/Raf-1, which in turn activate MAPK/ERK kinases referred to as MEKs. The MEKS in turn activate ERKs. The ERKS translocate to the cell nucleus where they activate transcription factors and thereby regulate cell proliferation. The modulation of these protein kinases produces neuroprotective and neuron-treating effects as does the modulation of the MAPK cascade. Examples of such kinases are mitogen-activated protein kinase 1 and 2, their homologues and isoforms, extracellular signal-regulated kinases (ERKs) their homologues and isoforms (ERK1, ERK2, ERK3, ERK4), and a group of kinases known as MAP/ERK kinases 1 and 2 or MEK1/2.

Exposure of cells to stress activates protein kinases by a variety of mechanisms. For example, ischemia, NMDA (N-methyl-D-aspartate) and amyloid peptides activate MAPK. Studies of functional roles of MAPKs in nerve tissue suggest that MAPK could be an important regulator of nerve cell death and plasticity. Thus, MAPK activation is required for hippocampal long-term potentiation (LTP).

In another aspect, the methods and compositions of the present invention can activate the ERK/MAP kinase pathway with nuclear translocation of p42 MAP kinase. In one embodiment, the present invention can be used to modulate, preferably increase, long-term memory. As shown in Example 12, a MAP kinase inhibitor completely antagonized the memory enhancing activity of intranasal Gilatide. Furthermore, [Ser(2)]exendin(1-9) (SEQ ID NO:1) increased MAP kinase activity in the hippocampus at 30 minutes. These data strongly support a model whereby activation of central GLP-1R by either local infusion of the full length peptide, or systemic administration of Gilatide or analogs leads to activation of the ERK/MAP kinase pathway with nuclear translocation of p42 MAP kinase, which is associated with long-term memory (N. Venable et al. Psychopharmacology 100, 215 (1990)).

As demonstrated in the Examples, GLP-1R contributes to learning and memory and can also mediate a neuroprotective phenotype. This is a receptor pathway that has not previously been implicated in learning and memory. Furthermore, the Examples show that GLP-1 and the 9-mer peptide, Gilatide, act via this pathway to produce potent memory-enhancing effects, similar to those observed with cognitive-enhancing agents in current clinical use. GLP-1R may therefore prove to be a promising target for therapeutic strategies directed towards neurodegenerative and cognitive disorders.

Gilatide and analogs can modulate memory disorder through interaction with GLP-1R to improve acquisition of memory or improve memory retention. To demonstrate the specificity of the effects of Gilatide on memory in vivo, parallel experiments were conducted in GLP-1R deficient (GLP-1R−/−) and wild-type mice as shown in Example 8. Consistent with mediation of the memory enhancing effects of Gilatide via GLP-1R, intranasal Gilatide did not enhance associative learning in the knockout mice but did in GLP-1R+/+ mice. GLP-R−/− mice were also tested in an associative learning paradigm: contextual fear conditioning. Compared to GLP-1R+/+ mice, GLP-1R−/− mice demonstrated a marked decrease in contextual fear conditioning. Gilatide improves acquisition of spatial learning in GLP-1R+/+ mice and significantly enhanced retention when tested 24 hours later. In contrast, GLP-1R−/− mice did not learn during the acquisition portion of the modified version of the MWM, and did not improve their performance following Gilatide administration. Moreover, Gilatide did not enhance retention of spatial learning in the GLP-1R−/− mice.

In another aspect of the invention, increasing GLP-1R levels in the hippocampus enhances learning. To demonstrate the role of the GLP-1R in learning and memory, two groups of rats were tested in the PA paradigm: pre-treatment with either Gilatide or vehicle. The hippocampus of each rat was processed and real-time quantitative RT-PCR was used to detect changes in GLP-1R mRNA. Training (vehicle pre-treatment) produced an increase in GLP-1R mRNA compared with sham-shocked controls, while pre-treatment with intranasal Gilatide decreased GLP-1R mRNA to the levels found in sham-shocked animals, and also significantly lowered the mRNA transcript levels compared to the vehicle-treated rats. In addition, rAAV vectors expressing control EGFP vector and GLP-1R were generated and injected into the hippocampus of rats as shown in Example 9. Rats that overexpressed GLP-1R showed marked enhancement in spatial learning, with reductions in both latency and distance traveled to locate the hidden platform compared to controls.

Thus, the compositions and methods of the present invention can improve hippocampal-dependent learning. In one embodiment, the compositions and methods of the present invention can improve associate learning. In another embodiment, the compositions and methods of the present invention can improve spatial learning. As shown in Example 4, the effects of centrally administered GLP-1 and Gilatide on associative and spatial learning, both of which are hippocampal dependent, were investigated using the passive avoidance (PA) and Morris Water Maze (MWM) paradigms in rats. GLP-1 and Gilatide administered intracerebroventricularly (i.c.v.) enhanced latency in the PA task, the effect being similar to that of vasopressin, a peptide previously shown to facilitate learning (D. DeWied, Nature 232, 58 (1971)). Consistent with its action as a GLP-1R antagonist, co-infusion of exendin(9-39) completely blocked the memory enhancing effects of GLP-1 and Gilatide, but not vasopressin. Assessment of the effects of i.c.v. GLP-1 and Gilatide on spatial learning in the MWM showed that both peptides significantly reduced distance traveled to locate the platform compared to control rats. Close examination of individual rat search patterns on day 5 of training showed that although GLP-1 and Gilatide-treated rats swam more slowly, they displayed a highly efficient search strategy compared to control rats, suggestive of enhanced spatial learning.

Clinically approved treatments for cognitive impairment act primarily on the cholinergic system. In Example 5, the effects of intranasal GLP-1 and Gilatide were compared with that of the cholinergic agonist arecoline on spatial learning in a modified version of the MWM. There were no differences between treatments in acquisition. However, Gilatide and arecoline, but not GLP-1, significantly reduced the latency for rats to locate the submerged platform in the retention trial.

Central administration of drugs poses major problems for translation to clinical applications. The potential for side-effects caused by systemic administration can be averted by nasal delivery (Born J, et al. Nat Neurosci. 2002 June; 5(6):514-6). As shown in Example 5, intranasal administration of Gilatide peptides and anologs, but not GLP-1, increased latency in the PA test to a similar extent as vasopressin. A scrambled peptide, containing the same 9 amino acids as SEQ ID NO:1, but in random order and not homologous to any known protein, produced similar latency as vehicle (VEH).

IV. Blood Glucose

In another aspect, the compositions and methods of the present invention can be used to modulate blood glucose. Gilatide and analogs can be used to modulate blood glucose through its effect on insulin production and/or secretion. Thus, the compositions and methods of the present invention can be used to prevent, delay onset or treat glucose-metabolism disorders.

A. Glucagon-Like Peptide 1

Glucagon-like peptide 1 (GLP-1) is a hormone derived from tissue-specific posttranslational processing of the proglucagon gene in intestinal L cells by post-translation processing of the preprogulcagon molecule into GLP-1(7-37) and GLP-1(7-36)amide, which are the biologically active forms of GLP-1. It is secreted into the circulation after oral food uptake and acts via a specific G-protein-coupled receptor (GLP-1R). It shares considerable amino acid sequence homology with glucagon, and this sequence is conserved in multiple vertebrate and invertebrate species, indicating an important role in normal physiology. Indeed, GLP-1 exerts effects on glucose-dependent insulin secretion, insulin biosynthesis, gastrointestinal motility, islet cell neogenesis, energy homeostasis and food intake (C. C. Tseng, et al. Am. J. Physiol. 76, E1049 (1999); D. J. Drucker, Endocrinology 142, 521 (2001); D. A. Stoffers et al., Diabetes 49, 741 (2000); M. D. Turton et al., Nature 379, 69 (1996)). GLP-1 and GLP-1R are also expressed in the brain including the hippocampus (S. L. C. Jin et al, J. Comp. Neurol. 271, 519 (1988); I. Merchenthaler, et al. J. Comp. Neurol. 403, 261 (1999); E. Alvarez et al. J. Neurochem. 66, 920 (1996)) a structure that shows considerable plasticity and is critical for several forms of learning and memory (E. R. Kandel, Science 294, 1030 (2001)). GLP-1 modulates gene expression and acts as a trophic and differentiation factor for pancreatic islet cells (J. Zhou et al. Diabetes 48, 2358 (1999); R. Perfetti et al. Endocrinology 141, 4600 (2000)). In one aspect, the present invention shows that Gilatide peptides and analogs as well as GLP-1 can act in the brain to influence hippocampal plasticity and facilitate learning.

GLP-1 upregulates glucose-induced insulin secretion and suppresses stomach acid secretion. Although derived from the same precursor as glucagon, GLP-1 has a distinct structure and is not active at the glucagon receptor. Several glucose disorders, such as non-insulin-dependent diabetes mellitus, are associated with a reduced stimulatory effect of GLP-1 on glucose-induced insulin secretion. GLP-1 can promote neogenesis and differentiation of pancreatic beta cells (Perfetti et al. Endocrinology, 2000, 141: 4600). GLP-1 is also involved in the regulation of food consumption (Thiele et al. (1997) Am J Physiol 272, R726-R730.) and that central administration of GLP-1 inhibits food and water intake in rats.

Glucagon is required for control of blood glucose levels. The peptide stimulates glycogenolysis and gluconeogenesis in liver, producing glucose for release into the bloodstream. It also causes lipolysis in liver and fat cells. Its major actions are therefore opposite from those of insulin, and it has a major role in the pathogenesis of diabetes. Glucagon has also occasionally been used to increase force and rate of contraction during acute cardiac failure. The sequence of glucagon is conserved across all mammalian species, and shares a limited sequence similarity with members of the VIP family (for example, 15 of the amino acids in glucagon are present in secretin) The glucagon receptor is expressed predominantly in liver. It is also found in adipose tissue and in heart. The sequence of the rat glucagon receptor is available through GenBank at accession numbers L04796 and M96674. The first transmembrane domain begins at about amino acid number 144.

Gilatides and analogs can be used in the treatment of glucose-metabolism disorders. Glucose-metabolism disorders include, but are not limited to, obesity, diabetes, anorexia nervosa, insulin resistance, glucose intolerance, hyerinsulinemia, Syndrome X, hypercholesterolemia, hyperlipoproteinemia, hypertriglyceridemia, atherosclerosis, and diabetic renal disease. For example, Gilatides and analogs are useful in the treatment of type 2 diabetes, a disease which is associated with insulin resistance in peripheral tissues and impaired glucose-stimulated insulin secretion from pancreatic beta-cells and elevated hepatic gluconeogenesis. Gilatides and analogs have been shown in Example 2 to increase insulin production. In addition, Gilatides and analogs can be used to increase pancreatic beta-cell neogenesis and glucose-dependent insulin secretion. Furthermore, the present invention can exert diverse insulinotropic actions on β-cells including stimulation of cAMP formation, insulin secretion, insulin biosynthesis, proinsulin gene expression, and inhibition of glucagon secretion. Gilatides and analogs can be used to control glucose homoeostasis.

B. Glucagon-Like Peptide 1 (GLP-1) Receptor

The sequence of the rat GLP-I receptor is available through GenBank at accession number M97797. The first transmembrane domain begins at about amino acid number 146. The extracellular region of GLP-1 receptor has been shown to bind GLP-1. GLP-1 receptors are coupled to multiple G proteins and diverse signaling pathways including cAMP, PKA, phospholipase C, PI-3 kinase and PKC, MAP kinases and intracellular Ca²⁺ (D. J. Drucker, et al. Proc. Natl. Acad. Sci. USA 84, 3434 (1987); Montrose-Rifizadeh, et al., Endocrinology 140, 1132 (1999); Wheeler, et al., Endocrinology 133, 57 (1993); Holz, et al. J. Biol. Chem. 270, 17749 (1995)). However, the contributions of each of these pathways for the many peripheral effects of GLP-1 remain poorly characterized, particularly those of most relevance to this study, that of neuroendocrine cell plasticity. However, islet cell differentiation in response to GLP-1 is blocked by a specific PKC inhibitor, with MAP kinase the likely downstream effector in this model (Zhou, et al., Diabetes 48, 2358 (1999)). Recent studies have shown that the ERK/MAP kinase cascade appears to be a conserved and critical pathway mediating cognition not only in multiple invertebrates and vertebrates, but also in humans (Weeber, et al. Neuron 33, 845 (2002)).

Example 12 shows that administration of Gilatide significantly increased pMAP kinase immunoreactivity in cytosolic and nuclear fractions of hippocampal samples taken following intranasal administration. In addition, the enhancement of associative learning by intranasal [Ser(2)]exendin(1-9) was completely blocked when PD98059, a specific MEK inhibitor that prevents subsequent ERK/MAPK activation, was administered to rats immediately after training in the PA paradigm but not when given before training.

In one aspect of the present invention, Gilatide peptides and analogs interact with the GLP-1 receptor. In one embodiment, the present invention demonstrates that the GLP-1R is involved in learning and memory processes. Human GLP-1R can be found in Genbank Accession Nos: U10037 and U01104. Heloderma suspectum exendin 4 can be found at Genbank Accession No: U77613. Both GLP-1 and a conserved 9-amino-acid N-terminal domain of the protein, Gilatide, enhance associative and spatial learning (See examples 3, 4, 7, 8, and 9). These effects were blocked by a GLP-1 antagonist. Moreover, upregulation of GLP-1R expression via hippocampal gene transfer enhances spatial and contextual learning. In addition, there is a corresponding upregulation of GLP-1R transcripts in response to training in an associative learning paradigm. Further evidence came from the studies performed in GLP-1R−/− mice (See Examples 8 and 9), which demonstrated that deficiency of this receptor results in decrements in the acquisition of spatial and contextual learning. Finally, [Ser(2)]exendin(1-9) administration did not enhance learning in GLP-1R−/− mice as it did in GLP-1R+/+ mice. In absence of any confounding motor or stress effects, these results show that GLP-1R as well as the interaction of Gilatide and analogs with GLP-1R play a role in learning and memory.

In one aspect of the invention, Gilatide and analogs increases insulin production. In one embodiment, the interaction of Gilatide and analogs with GLP-1R can modulate insulin production. The in vitro studies detailed in the Examples section show that the effects of Gilatide on insulinoma cells were comparable with those of GLP-1 and were blocked by the GLP-1 antagonist exendin (9-39). Example 2 demonstrates that Gilatide peptides and analogs can induce insulin production in rat insulinoma cells (RINm5f). Rat islet cells have been shown to behave like human islet cells with respect to glucose metabolism and insulinotropic action (Malaisse W. J. Diabetologia 2001 April; 44(4):393-406) and hence serve as a useful model of human diseases. In the central nervous system, GLP-1 regulates hypothalamic-pituitary function and GLP-1-activated circuits mediate the CNS response to aversive stimulation. In humans and experimental animals GLP-1 suppresses postprandial hyperglycemia and lowers blood glucose levels. GLP-1 receptor knockout mice (GLP-1R^{-/-}) exhibit hyperglycemia following an oral glucose challenge as a consequence of impaired insulin secretion. Gilatides and analogs interact with the GLP-1R, similar to GLP-1, resulting in the increase of insulin and, thus, decrease in blood glucose. The effects of Gilatide and analogs on insulin production is impaired in GLP-1 receptor knockout mice verifying that it interacts with GLP-1R.

Gilatide peptides and analogs can also modulate insulin through their interaction with pituitary adenylate cyclase-activating polypeptide (PACAP) receptors. Three types of receptors have been identified that belong to G-protein-coupled receptor superfamily with seven transmembrane domains that are expressed widely in tissues and cell lines, including pancreatic islets, insulin-secreting cell lines (MIN6, HIT-T15, and RINm5F), lung, brain, stomach, colon, and heart (Zhou et al. Curr Protein Pept Sci 2002 3(4):423-39).

In another embodiment, the interaction of Gilatide and analogs with GLP-1R can modulate memory disorders. As shown in the Examples section, Exendin (9-39), a GLP-1 antagonist, blocked the enhancement of associative learning by Gilatide and GLP-1. Administration of intranasal Gilatide before training in the PA paradigm resulted in downregulation of GLP-1R transcripts indicating a classical agonist effect. Together with the studies performed in GLP-1R−/− mice, these data suggest that Gilatide exerts its effects through the GLP-1R.

Following intranasal administration of Gilatide and GLP-1, only Gilatide facilitated learning, while a scrambled peptide with the same residues as Gilatide, but synthesized in random order, was inactive. Consistent with its insulinotropic action, GLP-1 potently decreased fasting glucose levels when delivered systemically, whereas Gilatide did not. Moreover, intranasal GLP-1 is anxiogenic which is also related to modulation of blood glucose. These peripheral metabolic effects of GLP-1 may have surmounted any potential central effects, as hypoglycemia is known to be associated with impaired learning (A. C. Santucci et al. Behav. Neural. Biol. 53, 321 (1990)) and is anxiogenic.

V. Neuroprotection

In yet another aspect, the compositions and methods of the present invention can be used to prevent, delay onset or treat neurological disorders. In one embodiment, the methods of the present invention can be used to protect a subject against neurotoxins.

The use of kainic acid to induce seizures and neuropathological changes in a subject is a well-known model that has been useful in evaluating treatment that can prevent seizure-induced structural damage (Leite J P et al. Epilepsy Res 2002 50(1-2):93-103). Kainic acid induces changes that mimic human temporal lobe epilepsy, generalized motor seizures, as well as the pattern of cell damage caused by seizures in the hippocampus (Miettinen R et al. Brain Res 1998; 813:9-17). Intranasal administration of Gilatide (SEQ ID NO:1), but not scrambled peptide, to rats led to lower rates of KA-induced apoptosis among hippocampal neurons (See Example 13). In addition, Example 13 shows that GLP-1R−/− mice were more susceptible to the potent neurotoxin, kainic acid (KA), resulting in more (KA)-induced seizures and neuronal degeneration in the hippocampus than wild-type mice. Activation of GLP-1R facilitates cellular repair and neogenesis in the periphery, as evidenced by GLP-1-induced pancreatic cell differentiation and neogenesis. Previous studies have demonstrated increased GLP-1R expression in response to penetrating brain trauma (J. A. Chowen, et al., Neuropeptides 33, 212 (1999)). Moreover, GLP-1 facilitates neurite outgrowth and potentiates NGF-initiated cellular differentiation in vitro. Example 13 provides evidence that GLP-1R signaling may be an important pathway in neuronal plasticity and neuroprotection. In addition, this study provides evidence that Gilatide peptides and analogs reduce cell death caused by seizures, delay onset of seizures and prevent seizures through its interaction with GLP-1R.

Apoptotic cell death contributes to brain damage following seizures. Experimental evidence has shown that many degenerating neurons within the brain display morphological changes associated with apoptosis following prolonged seizures resulting from systemic or local injections of seizure-inducing neurotoxins, i.e., kainic acid and pilocarpine. Recent studies indicate that a single seizures could lead to apoptotic neuronal death. (Bengzon J. et al. Prog Brain Res 2002; 135:111-9). In one aspect of the invention, Gilatide peptides and analogs can delay or prevent seizures. Example 13 illustrates that administering Gilatide peptides prolonged the time between administering KA and the onset of the seizure. In addition, Gilatide administion reduced the number of apoptotic cells in response to KA-induced seizures. Thus, Gilatide peptides and analogs have a neuroprotective effect. The neuroprotective effect includes, but is not limited to, improved neuronal function, improved synaptic plasticity, protection from neurotoxins, decreased neuronal cell loss and glial cell loss, and decreased cell degeneration. In another embodiment, Gilatide and analogs is neurotrophic when adminstered to a subject.

The methods and compositions of the present invention can also improve synaptic plasticity. Interventions that improve synaptic plasticity may be associated with neuroprotective efficacy. Both environmental enrichment and cognitive enhancing agents can lead to an increase in the brain's resistance to insults. In addition, molecules that facilitate learning and memory can also help to protect the CNS against various insults. For example, GLP-1 promotes neogenesis and differentiation of pancreatic beta cells (Perfetti et al. Endocrinology, 2000, 141: 4600), suggesting that it may also have neurotrophic and neuroprotective activity. Therefore, the effects of the potent neurotoxin, kainic acid (KA), which produces excessive hippocampal excitation and cell loss, particularly in the CA3 subregion when administered systemically, were investigated in GLP-1R+/+ and GLP-1R−/− mice. Significantly lower seizure latency times were observed in GLP-1R−/− compared to GLP-1R+/+ mice. Moreover, seizure severity was greater in the GLP-1R−/− mice (See Example 13). These results suggest that the GLP-1R and the interaction of Gilatide and analogs with GLP-1R play an important role in neuroprotection.

Parallel experiments were done to determine the effects of Gilatides and analogs on KA-induced apoptosis. Compared to the scrambled peptide, Gilatide peptides and analogs significantly decreases KA-induced apoptosis in the CA3 region of the hippocampus, as measured by the number of TdT-mediated dUTP nick end labeling (TUNEL)-positive cells (See Example 13).

The present invention demonstrates that GLP-1 and peptides comprising SEQ ID NO:1 or modified analogs thereof can impart neuroprotection when administered to a subject. SEQ ID NO:1 is homologous to a conserved domain in the glucagon/GLP-1 family. The hippocampus is particularly vulnerable to neuronal loss associated with epilepsy, stroke, seizures, brain ischemia and neurodegenerative disorders. As demonstrated in Examples 13, GLP-1 and peptides comprising SEQ ID NO:1 and/or analogs thereof can play a the role in neuroprotection and can delay onset of seizures. The neuroprotective effects of Gilatide and analogs demonstrated in Example 13 can be useful in the treatment or prophylaxis of neurological disorders including, but not limited to, head injury, spinal cord injury, seizures, stroke, dementia, memory loss, attention deficit disorder (ADD), epilepsy, and ischemia. Neurological disorders also include neurodegenerative diseases. Neurodegeneration can occur in any area of the brain of a subject and is seen with many disorders including, but not limited to, Amyotrophic Lateral Sclerosis (ALS), multiple sclerosis, Huntington's disease, Parkinson's disease and Alzheimer's disease.

VI. Gilatide Peptides and Analogs.

In one embodiment, Gilatide peptides comprising SEQ ID NO:1 and active analogs thereof can be synthesized with amino-acid and non-amino acid residues that are capable of improving pharmaceutical relevant properties, such as, but not limited to, solubility, stability, and lipophilicity. In a preferred embodiment, Gilatide can be synthesized with a stearic acid residue added to the N terminus to improve lipophilicity and a serine substituted for glutamine in position 2 to improve peptide stability, as this residue is critical for dipeptidyl-peptidase IV mediated degradation (B. Gallwitz et al., Regul. Pept. 86, 103 (2000)). Additional amino acid and non-amino acid substitutions are well-known in the art and are discussed above. Biological activity of Gilatide peptides and analogs can be confirmed as described in the Examples section.

The present invention relates to Gilatide and to variations of the Gilatide peptide that show the biological activity or function of Gilatide. This biological activity or function may include an improved activity or a decreased undesirable activity. Functional assays for Gilatide are described below in the Examples section. Such variants of Gilatide include functional analogs, derivatives, fragments, and mimetics of Gilatide. The invention further includes methods for selecting functional analogs, fragments, and mimetics of Gilatide from a collection of randomly obtained or rationally designed candidate compounds. Compounds selected by the process described herein will retain the biological activity or function of Gilatide. Nucleic acids encoding Gilatide and fragments, analogs, derivatives, and mimetics thereof are also provided.

The fragments, derivatives, analogs, or mimetics of the Gilatide peptide may be: (1) one in which one or more of the amino acid residues are substituted with a conserved or non-conserved amino acid residue; (2) one in which one or more of the amino acid residues includes a substituent group; (3) one in which the mature peptide is fused with another compound, such as a compound to increase the half-life of the peptide (for example, polyethylene glycol); (4) one in which the additional amino acids are fused to the mature peptide, such as a leader or secretory sequence or a sequence that is employed for purification of the mature peptide or a propeptide sequence; or (5) one which comprises fewer or greater amino acid residues than has SEQ. ID. NO:1 and yet still retains activity characteristics of Gilatide. Such fragments, derivatives, analogs, and mimetics are deemed to be within the scope of those skilled in the art from the teachings herein.

A. Identification of Gilatide Analogs

One skilled in the art may prepare such fragments, derivatives, analogs, or mimetics of the Gilatide peptide by modifying the native sequence by resultant single or multiple amino acid substitutions, additions, or deletions. These changes are preferably of a minor nature, such as conservative amino acid substitutions, that do not significantly affect the folding or activity of the peptide. For instance, one polar amino acid, such as threonine, may be substituted for another polar amino acid, such as serine; or one acidic amino acid, such as asparatic acid, may be substituted for another acidic amino acid, such as glutamic acid; or a basic amino acid, such as lysine, arginine, or histidine, may be substituted for another basic amino acid; or a non-polar amino acid, such as alanine, leucine or isoleucine, may be substituted for another non-polar amino acid. Guidance concerning which amino acid changes are likely to be phenotypically silent can be found in Bowie, J. U., et al., “Deciphering the Message in Protein Sequences: Tolerance to Amino Acid Substitutions,” Science 247:1305-1310 (1990). Of course, the number of amino acid substitutions a skilled artisan would make depends on many factors.

Moreover, Gilatide amino acids that are essential for function can be identified and variations can be made using methods known in the art, such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)) cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce Gilatide variant DNA.

One well-known method for identifying Gilatide amino acid residues or regions for mutagenesis is known as “alanine scanning mutagenesis.” See, e.g., Cunningham and Wells, Science (1989) 244:1081-1085. In this method, an amino acid residue or group of target residues are identified (e.g., charged residues such as Arg, Asp, His, Lys, and Glu) and replaced by a neutral or negatively charged amino acid (most preferably alanine or polyalanine) to affect the interaction of the amino acids with the surrounding aqueous environment in or outside the cell. Those domains demonstrating functional sensitivity to the substitutions are refined by introducing additional or alternate residues at the sites of substitution. Thus, the target site for introducing an amino acid sequence variation is determined, alanine scanning or random mutagenesis is conducted on the corresponding target codon or region of the DNA sequence, and the expressed Gilatide analogs are screened for the optimal combination of desired activity and degree of activity.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, Science, 244: 1081-1085 (1989)). Alanine is also typically preferred because it is the most common amino acid. Further, it is frequently found in both buried and exposed positions (Creighton, The Proteins, (W. H. Freeman & Co., N.Y.); Chothia, J. Mol. Biol., 150:1 (1976)). If alanine substitution does not yield adequate amounts of variant, an isoteric amino acid can be used.

Initially, sites can be substituted in a relatively conservative manner. If such substitutions result in a change in biological activity, then more substantial changes (exemplary substitutions) are introduced, and/or other additions or deletions may be made, and the resulting products screened for activity. Non-conservative substitutions will entail exchanging a member of one of these classes for another class. Such substituted residues also may be introduced into the conservative substitution sites or, more preferably, into the remaining (non-conserved) sites.

Peptides of the present invention can be prepared in any suitable manner. Such peptides include isolated naturally occurring peptides, recombinantly produced peptides, synthetically produced peptides, or peptides produced by a combination of these methods. Means for preparing such peptides are well known in the art.

Peptides of the instant invention can be identified by screening a large collection, or library, of random peptides or peptides of interest. Peptide libraries include, for example, tagged chemical libraries comprising peptides and peptidomimetic molecules. Peptide libraries also comprise those generated by phage display technology. Phage display technology includes the expression of peptide molecules on the surface of phage as well as other methodologies by which a protein ligand is or can be associated with the nucleic acid that encodes it. Methods for the production of phase display libraries, including vectors and methods of diversifying the population of peptides that are expressed, are well known in the art (see, for example, Smith & Scott, Methods Enzymol. 217:228-257 (1993); Scott & Smith, Science 249:386-390 (1990); and Huse, WO 91/07141 and WO 91/07149, each of which is incorporated herein by reference). These or other well known methods can be used to produce a phage display library, from which the displayed peptides can be cleaved and assayed for activity, for example, using the methods disclosed infra. If desired, a population of peptides can be assayed for activity, and an active population can be subdivided and the assay repeated in order to isolate an active peptide from the population. Other methods for producing peptides useful in the invention include, for example, rational design and mutagenesis based on the amino acid sequences of active fragments of Gilatide.

An active analog of Gilatide, useful in the invention, can be isolated or synthesized using methods well known in the art. Such methods include recombinant DNA methods and chemical synthesis methods for production of a peptide. Recombinant methods of producing a peptide through expression of a nucleic acid sequence encoding the peptide in a suitable host cell are well known in the art and are described, for example, in Sambrook et al., Molecular Cloning: A Laboratory Manual, 2^{nd} Ed, Vols 1 to 3, Cold Spring Harbor Laboratory Press, New York (1989), which is incorporated herein by reference.

B. Generation of Gilatide and Analogs

Gilatide peptides or analogs thereof useful in the invention also can be produced by chemical synthesis, for example, by the solid phase peptide synthesis method of Merrifield et al., J. Am. Chem. Soc. 85:2149 (1964), which is incorporated hereby by reference. Standard solution methods well known in the art also can be used to synthesize a peptide useful in the invention (see, for example, Bodanszky, Principles of Peptide Synthesis, Springer-Verlag, Berlin (1984) and Bodanszky, Peptide Chemistry, Springer-Verlag, Berlin (1993), each of which is incorporated herein by reference). A newly synthesized peptide can be purified, for example, by high performance liquid chromatography (HPLC), and can be characterized using, for example, mass spectrometry or amino acid sequence analysis.

In addition, active analogs, derivatives, fragments or mimetics of Gilatide can be synthesized by use of a peptide synthesizer. Furthermore, if desired, non-classical amino acids or chemical amino acid analogs can be introduced as a substitution or addition into the Gilatide sequence. Non-classical amino acids include but are not limited to the D-isomers of the common amino acids, α-amino isobutyric acid, 4 amino-butyric acid, Abu, 2-amino butyric acid, γ-Abu, ε-Ahx, 6-amino hexanoic acid, Aib, 2-amino isobutyric acid, 3-amino propionic acid, omithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer aminoacids such as β-methyl amino acids, C-α-methyl amino acids, N-α-methyl amino acids, and amino acid analogs in general. Furthermore, the amino acid can be D (dextrorotary) or L (levorotary).

It is understood that limited modifications can be made to an active analog, derivative, fragment or mimetic of Gilatide without destroying its biological function. Thus, a modification of a functional analog, derivative, fragment or mimetic of Gilatide that does not destroy its activity or function is within the definition of a functional analog, derivative, fragment or mimetic of Gilatide. A modification can include, for example, an addition, deletion, or substitution of amino acid residues; a substitution of a compound that mimics amino acid structure or function; and addition of chemical moieties such as amino or acetyl groups.

A particularly useful modification is one that confers, for example, increased stability. For example, incorporation of one or more D-amino acids or substitution or deletion of lysine can increase the stability of an active analog, derivative, fragment or mimetic of Gilatide by protecting against peptide degradation. The substitution or deletion of a lysine residue confers increased resistance to trypsin-like proteases, as is well known in the art (Partridge, Peptide Drug Delivery to the Brain, Raven Press, New York, 1991). These substitutions increase stability and, thus, bioavailability of peptides, but do not affect activity.

A useful modification also can be one that promotes peptide passage across the blood-brain barrier, such as a modification that increases lipophilicity or decreases hydrogen bonding. For example, a tyrosine residue added to the C-terminus of a peptide may increase hydrophobicity and permeability to the blood-brain barrier (see, for example, Banks et al., Peptides 13:1289-1294 (1992), which is incorporated herein by reference, and Pardridge, supra, 1991). A chimeric peptide-pharmaceutical that has increased biological stability or increased permeability to the blood-brain barrier, for example, also can be useful in the method of the invention.

Using this information, a variety of recombinant DNA vectors are provided which are capable of providing, in reasonable quantities, Gilatide peptides and analogs. Additional recombinant DNA vectors of related structure that code for proteins comprising key structural features identified herein, such as functional Gilatide analogs, can be produced from or identified with the Gilatide nucleotide sequence (SEQ ID NO:2) using standard techniques of recombinant DNA technology. Likewise, proteins of the same family from other sources can also be identified with the Gilatide nucleotide sequence and corresponding protein described herein. Transformants expressing Gilatide or homologs thereof have been produced as an example of this technology. The newly discovered sequence and structural information can be used, through transfection of eukaryotic cells, to prepare polypeptides retaining bioactivity, as well as fusion proteins which include the Gilatide polypeptide.

Since there is a known and definite correspondence between amino acids in a peptide and the DNA sequence that codes for the peptide, the DNA sequence of a DNA or RNA molecule coding for Gilatide peptide (or any of the modified peptides) can be use to derive the amino acid sequence, and vice versa. Such a sequence of nucleotides encoding a Gilatide peptide protein is shown in SEQ ID NO: 2, along with the corresponding amino acid sequence (shown also in SEQ ID NO: 1). Complementary trinucleotide DNA sequences having opposite strand polarity are functionally equivalent to the codons of SEQ ID NO: 2, as is understood in the art. An important and well known feature of the genetic code is its redundancy, whereby, for most of the amino acids used to make proteins, more than one coding nucleotide triplet may be employed. Therefore, a number of different nucleotide sequences may code for a given amino acid sequence. Such nucleotide sequences are considered functionally equivalent since they can result in the production of the same amino acid sequence in all-organisms, although certain strains may translate some sequences more efficiently than they do others. Occasionally, a methylated variant of a purine or pyrimidine may be found in a given nucleotide sequence. Such methylations do not affect the coding relationship in any way. The equivalent codons are well known in the art (See for example, Voet and Voet Biochemistry John Wiley & Sons, Inc (1995)).

Since the DNA sequence of the coding region of the gene has been fully identified, it is possible to produce a nucleic acid encoding a Gilatide, or portion thereof, entirely by synthetic chemistry, after which the gene can be inserted into any of the many available DNA vectors using known techniques of recombinant DNA technology. Thus the present invention can be carried out using reagents, plasmids, microorganism, and eukaryotic cells which are freely and readily available.

Various methods of chemically synthesizing polydeoxynucleotides are known, including solid-phase synthesis which, like peptide synthesis, has been fully automated in commercially available DNA synthesizers (See the Itakura et al. U.S. Pat. No. 4,598,049; the Caruthers et al. U.S. Pat. No. 4,458,066; and the Itakura U.S. Pat. Nos. 4,401,796 and 4,373,071). For example, nucleotide sequences greater than 100 bases long could be readily synthesized in 1984 on an Applied Biosystems Model 380A DNA Synthesizer as evidenced by commercial advertising of the same (e.g., Genetic Engineering News, November/December 1984, p. 3). Such oligonucleotides can readily be spliced using, among others, the techniques described later in this application to produce any nucleotide sequence described herein. For example, relatively short complementary oligonucleotide sequences with 3′ or 5′ segments that extend beyond the complementary sequences can be synthesized. By producing a series of such short segments, with “sticky” ends that hybridize with the next short oligonucleotide, sequential oligonucleotides can be joined together by the use of ligases to produce a longer oligonucleotide that is beyond the reach of direct synthesis. Furthermore, automated equipment is also available that makes direct synthesis of any of the peptides disclosed herein readily available. Such equipment provides ready access to the peptides of the invention, either by direct synthesis or by synthesis of a series of fragments that can be coupled using other known techniques.

In addition to the specific peptide sequence shown in SEQ. ID No. 1, other peptides based on this sequence and representing variations thereof can have similar biological activities of Gilatide. Additional exogenous amino acids can be present at either or both terminal ends of the core protein or its truncations. These added sequences can, for example, facilitate purification, or be used for in the generation of fusion proteins having novel activities.

Within the portion of the molecule containing the Gilatide sequence, replacement of amino acids is more restricted in order that biological activity can be maintained. However, variations of the previously mentioned peptides and DNA molecules are also contemplated as being equivalent to those peptides and DNA molecules that are set forth in more detail, as will be appreciated by those skilled in the art. For example, it is reasonable to expect that an isolated replacement of a leucine with an isoleucine or valine, an aspartate with a glutamate, a threonine with a serine, or a similar replacement of an amino acid with a structurally related amino acid (i.e. conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cystine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, senne, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methoinine. (see, for example, Biochemistry, 2nd ed, Ed. by L. Stryer, WH Freeman and Co.: 1981). Whether a change in the amino acid sequence of a peptide results in a functional Gilatide sequence can readily be determined by assessing the ability of the corresponding DNA encoding the peptide to produce this peptide in a form containing a Gilatide peptide when expressed by eukaryotic cells. Peptides in which more than one replacement has taken place can readily be tested in the same manner.

DNA molecules that code for such peptides can easily be determined from the list of codons and are likewise contemplated as being equivalent to the DNA sequence of SEQ ID NO: 2. In fact, since there is a fixed relationship between DNA codons and amino acids in a peptide, any discussion in this application of a replacement or other change in a peptide is equally applicable to the corresponding DNA sequence or to the DNA molecule, recombinant vector, transformed microorganism, or transfected eukaryotic cells in which the sequence is located (and vice versa). Codons can be chosen for use in a particular host organism in accordance with the frequency with which a particular codon is utilized by that host, if desired, to increase the rate at which expression of the peptide occurs.

In addition to the specific nucleotides given in SEQ. ID NO: 2 and analogs thereof DNA (or corresponding RNA) molecules of the invention can have additional nucleotides preceding or following those that are specifically listed. For example, a poly-adenylation signal sequence can be added to the 3′-terminus, nucleotide sequences corresponding to a restriction endonuclease sites can be added so as to flank the recombinant gene, and/or a stop codon can be added to terminate translation and produce truncated forms of the proteins. Additionally, DNA molecules containing a promoter region or other transcriptional control elements, upstream or downstream of the recombinant gene can be produced. All DNA molecules containing the sequences of the invention will be useful for at least one purpose since all can minimally be fragmented to produce oligonucleotide probes and be used in the isolation of additional DNA from biological sources.

By “purified”, it is meant, when referring to a peptide or DNA or RNA sequence, that the indicated molecule is present in the substantial absence of other biological macromolecules of the same type, such as other proteins. The term “purified” as used herein preferably means at least 95% by weight, more preferably at least 99% by weight, and most preferably at least 99.8% by weight, of biological macromolecules of the same type present (but water, buffers, and other small molecules, especially molecules having a molecular weight of less than 1000, can be present). The term “pure” as used herein preferably has the same numerical limits as “purified” immediately above. The term “isolated” as used herein refers to a peptide, DNA, or RNA molecule separated from other peptides, DNAs, or RNAs, respectively, that are present in the natural source of the macromolecule. “Isolated” and “purified” do not encompass either natural materials in their native state or natural materials that have been separated into components (e.g., in an acrylamide gel) but not obtained either as pure substances or as solutions.

Two protein sequences (or peptides derived from them of at least 9 amino acids in length) are homologous (as this term is preferably used in this specification) if they have an alignment score of >5 (in standard deviation units) using the program ALIGN with the mutation data matrix and a gap penalty of 6 (or greater). See Dayhoff, M. O., in Atlas of Protein Sequence and Structure, 1972, volume 5, National Biomedical Research Foundation, pp. 101-110, and Supplement 2 to this volume, pp. 1-10. The two sequences (or parts thereof—probably at least 30 amino acids in length) are more preferably homologous if their amino acids are greater than or equal to 50% identical when optimally aligned using the ALIGN program mentioned above. Two DNA sequences (or a DNA and RNA sequence) are homologous if they hybridize to one another using nitrocellulose filter hybridization (one sequence bound to the filter, the other as a ³²P-labeled probe) using hybridization conditions of 40-50% formamide, 37°-42° C., 4×SSC and wash conditions (after several room temperature washes with 2×SSC, 0.05% SDS) of stringency equivalent to 37° C. with 1×SSC, 0.05% SDS.

The phrase “replaced by” or “replacement” as used herein does not necessarily refer to any action that must take place, but rather to the peptide that exists when an indicated “replacement” amino acid is present in the same position as the amino acid indicated to be present in a different formula (e.g., when leucine is present at a particular amino acid position instead of isoleucine).

Salts of any of the macromolecules described herein will naturally occur when such molecules are present in (or isolated from) aqueous solutions of various pHs. All salts of peptides and other macromolecules having the indicated biological activity are considered to be within the scope of the present invention. Examples include alkali, alkaline earth, and other metal salts of carboxylic acid residues, acid addition salts (e.g., HCl) of amino residues, and zwitter ions formed by reactions between carboxylic acid and amino residues within the same molecule.

The invention has specifically contemplated each and every possible variation of peptide or nucleotide that could be made by selecting combinations based on the amino acid and nucleotide sequences disclosed in SEQ. ID. Nos: 1 and 2, and possible conservative amino acid substitutions and the choices of codons and all such variations are to be considered as being specifically disclosed.

Included within the scope of the invention are active analogs, derivatives, fragments or mimetics of Gilatide that are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogens bromide, trypsin, chymotrypsin, papain, V8 protease, NaBH₄; acetylation, formylation, oxidation, reduction; metabolic synthesis in the presence of tunicamycin; etc. The terms “Gilatide” and/or “Gilatide peptide” as used herein are intended to encompasses not only the amino acid sequence (SEQ ID NO: 1) but also these various derivatives and modifications.

Moreover, the peptide of the present invention can be a chimeric, or fusion, protein comprising Gilatide or an analog, derivative, fragment, or mimetic thereofjoined at its amino- or carboxy-terminus via a peptide bond to an amino acid sequence of a different protein. In one embodiment, such a chimeric protein is produced by recombinant expression of a nucleic acid encoding the protein. Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer.

VII. Therapeutic Uses

In one aspect of the invention, Gilatide and analogs can be used for the therapeutic and prophylactic treatment of neurological disorders. Neurological disorder can be associated with neuronal loss or dysfunction, including, but not limited to, Parkinson's Disease, Alzheimer's Disease, Huntington's Disease, ALS, stroke, epilepsy, ADD, and neuropsychiatric syndromes. In one embodiment, the neurological disorder is a neurodegenerative disorder. In another embodiment, the neurological disorder is selected from the group comprising seizures, strokes, brain ischemia, and epilepsy.

Compounds of the instant invention are administered therapeutically (including prophylactically): (1) in diseases, disorders, or conditions involving neuronal loss or dysfunction, including, but not limited to, Parkinson's Disease, Alzheimer's Disease, Huntington's Disease, ALS, stroke, ADD, and neuropsychiatric syndromes; or (2) in diseases, disorders, or conditions wherein in vitro (or in vivo) assays indicate the utility of the peptides of the present invention.

Alzheimer's disease (AD) is a degenerative brain disease, the incidence of which rapidly increases with advancing age. Certain populations of brain cells progressively die, particularly but by no means exclusively those using acetylcholine as a neurotransmitter. Recently modern imaging techniques have revealed how the medial temporal lobe area, which contains the hippocampus (a vital structure for learning and memory generally in humans and for certain types of spatial learning in animals) progressively shrinks as Alzheimer's disease runs its course. The principle symptoms of Alzheimer's disease are steadily progressive loss of cognitive faculties such as memory (particularly recent episodic memories), problems with language and speech such as difficulty in finding the right words, and attention. Multi-infarct dementia, the most common other form of dementia, often presents a similar clinical picture but as it is due to a series of small strokes its progression is more stepwise. In one aspect of the invention, Gilatide peptides or functional analogs can delay onset, amerliorate the symptoms, or treat Alzheimer's disease.

In another aspect of the invention, Gilatide and analogs can be used for the the therapeutic and prophylactic treatment of memory disorders. In another aspect of the inveniton, Gilatide and analogs can be used to improve learning and cognition. Memory disorder refers to a diminished mental registration, retention or recall of past experiences, knowledge, ideas, sensations, thoughts or impressions. Memory disorder may affect short and/or long-term information retention, facility with spatial relationships, memory (rehearsal) strategies, and verbal retrieval and production. The term memory disorder is intended to include dementia, slow learning and the inability to concentrate. Common causes of a memory disorder are age, severe head trauma, brain anoxia or ischemia, alcoholic-nutritional diseases, drug intoxications and neurodegenerative diseases. For example, a memory disorder is a common feature of neurodegenerative diseases, such as Alzheimer's disease (i.e. Alzheimer-type dementia). Memory disorders also occur with other kinds of dementia such as AIDS Dementia; Wernicke-Korsakoff's related dementia (alcohol induced dementia); age related dementia, multi-infarct dementia, a senile dementia caused by cerebrovascular deficiency, and the Lewy-body variant of Alzheimer's disease with or without association with Parkinson's disease. Loss of memory is also a common feature of brain-damaged patients. Non-limiting examples of causes of brain damage which may result in a memory disorder include stroke, seizure, an anaesthetic accident, ischemia, anoxia, hypoxia, cerebral edema, arteriosclerosis, hematoma or epilepsy; spinal cord cell loss; and peripheral neuropathy, head trauma, hypoglycemia, carbon monoxide poisoning, lithium intoxication, vitamin (B1, thiamine and B12) deficiency, or excessive alcohol use.

In yet another aspect of the invention, Gilatide and analogs can be used for the the therapeutic and prophylactic treatment of glucose-metabolism disorders. In one embodiment, administration of Gilatide or function analogs can modulate blood glucose. Gilatide or functional analogs can modulate the secretion of insulin leading to the modulation of blood glucose. Glucose-metabolism disorder is intended to refer to any disorder relating to glucose uptake or release, as well as, insulin expression, production, secretion, or usage. The glucose-metabolism disorder can be selected from, but not limited to, the group consisting of obesity, diabetes, anorexia nervosa, insulin resistance, hyperglycemia, glucose intolerance, hyerinsulinemia, Syndrome X, hypercholesterolemia, hyperlipoproteinemia, hypertriglyceridemia, atherosclerosis, and diabetic renal disease.

A. Delivery Methods

Various delivery systems are known and are used to administer a therapeutic of the invention, e.g., encapsulation in liposomes, microparticles, microcapsules, expression by recombinant cells, receptor-mediated endocytosis (see, e.g., Wu & Wu, J. Biol. Chem. 265:4429-4432, 1987), construction of a therapeutic nucleic acid as part of a retroviral or other vector, etc. Methods of introduction include, but are not limited to, intradermal, intramuscular, intraperitoneal, intravenous, subcutaneous, intranasal, and oral routes. The compounds are administered by any convenient route, for example by infusion or bolus injection, by absorption through epithelial or mucocutaneous linings (e.g., oral mucosa, rectal, and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers.

In an embodiment where the therapeutic is a nucleic acid encoding a Gilatide peptide or analog therapeutic the nucleic acid is administered in vivo to promote expression of its encoded Gilatide peptide by constructing it as part of an appropriate nucleic acid expression vector and administering it so that it becomes intracellular, e.g., by use of retroviral vector (see U.S. Pat. No. 4,980,286), or by direct injection, or by use of microparticle bombardment (e.g., a gene gun; Biolistic, Dupont) or coating with lipids or cell-surface receptors or transfecting agents, or by administering it in linkage to a homeobox-like peptide that is known to enter the nucleus (see e.g., Joliot et al., Proc. Natl. Acad. Sci., U.S.A. 88:1864-1868, 1991), etc., supra. Alternatively, a nucleic acid therapeutic can be introduced intracellularly and incorporated within host cell DNA for expression by homologous recombination.

Other methods for improving the delivery and administration of the pharmacological agent of the present invention include means for improving the ability of the pharmacological agent to cross membranes, and in particular, to cross the blood-brain barrier. In one embodiment, the pharmacological agent can be modified to improve its ability to cross the blood-brain barrier, and in an alternative embodiment, the pharmacological agent can be co-administered with an additional agent, such as for example, an anti-fungal compound, that improves the ability of the pharmacological agent to cross the blood-brain barrier. Alternatively, precise delivery of the pharmacological agent into specific sites of the brain, can be conducted using stereotactic microinjection techniques. For example, the subject being treated can be placed within a stereotactic frame base (MRI-compatible) and then imaged using high resolution MRI to determine the three-dimensional positioning of the particular region to be treated. The MRI images can then be transferred to a computer having the appropriate stereotactic software, and a number of images are used to determine a target site and trajectory for pharmacological agent microinjection. The software translates the trajectory into three-dimensional coordinates that are precisely registered for the stereotactic frame. In the case of intracranial delivery, the skull will be exposed, burr holes will be drilled above the entry site, and the stereotactic apparatus used to position the needle and ensure implantation at a predetermined depth. The pharmacological agent can be delivered to regions, such as the cells of the spinal cord, brainstem, or brain that are associated with the disease or disorder. For example, target regions can include the medulla, pons, and midbrain, cerebellum, diencephalon (e.g., thalamus, hypothalamus), telencephalon (e.g., corpus stratium, cerebral cortex, or within the cortex, the occipital, temporal, parietal or frontal lobes), or combinations, thereof.

One skilled in the art can readily assay the ability of an active analog, derivative, fragment or mimetic of Gilatide to cross the blood-brain barrier in vivo, for example using a model of the blood-brain barrier based on a brain microvessel endothelial cell culture system, for example as described in Bowman et al., Ann. Neurol. 14:396-402 (1983) or Takahura et al., Adv. Pharmacol. 22:137-165 (1992), each of which is incorporated herein by reference.

B. Genetic Engineering

The invention also provides a method of transplanting into the subject a cell genetically modified to express and secrete a peptide of the present invention. Transplantation can provide a continuous source of peptide of the instant invention and, thus, sustained treatment. For a subject suffering from neuronal loss or dysfunction, such a method has the advantage of obviating or reducing the need for repeated administration of an active peptide.

A nucleic acid encoding Gilatide or an analog thereof can be expressed under the control of one of a variety of promoters well known in the art, including a constitutive promoter or inducible promoter. See, for example, Chang, supra, 1995. A particularly useful constitutive promoter for high level expression is the Moloney murine leukemia virus long-terminal repeat (MLV-LTR), the cytomegalovirus immediate-early (CMV-IE) or the simian virus 40 early region (SV40).

Using methods well known in the art, a cell can readily be transfected with an expression vector containing a nucleic acid encoding a peptide of the instant invention (Chang, Somatic Gene Therapy, CRC Press, Boca Raton (1995), which is incorporated herein by reference). Following transplantation into the brain, for example, the transfected cell expresses and secretes an active peptide. The cell can be any cell that can survive when transplanted and that can be modified to express and secrete Gilatide or an analog, derivative, fragment, or mimetic thereof.

The cells can also be xenogenic, where the cells are derived from a mammalian species that are different from the subject. For example the different cells can be derived from mammals such as monkeys, dogs, cats, mice, rats, cows, horses, pigs, goats and sheep. Such cells can be obtained by appropriate biopsy or upon autopsy. Cadavers may be used to provide a supply of cells. The isolated cells are preferably autologous cells, obtained by biopsy from the subject. For example, a biopsy of cells from the arm, forearm, or lower extremities, from the area treated with local anaesthetic with a small amount of lidocaine injected subcutaneously, and expanded in culture. The biopsy can be obtained using a biopsy needle, a rapid action needle which makes the procedure quick and simple. The small biopsy core can then be expanded and cultured as known in the art. In practice, the cell should be immunologically compatible with the subject. For example, a particularly useful cell is a cell isolated from the subject to be treated, since such a cell is immunologically compatible with the subject. Cells from relatives or other donors of the same species can also be used with appropriate immunosuppression. Alternatively stem cells may be used.

In one aspect of the invention, the stem cells can be genetically engineered to constitutively or transiently produce Gilatide or analogs thereof. Stem cells can be derived from a human donor, e.g., pluripotent hematopoietic stem cells, embryonic stem cells, adult somatic stem cells, myeloid-origin stem cells and the like. The stem cells can be cultured in the presence of combinations of polypeptides, recombinant human growth and maturation promoting factors, such as cytokines, lymphokines, colony stimulating factors, mitogens, growth factors, and maturation factors, so as to differentiate into the desired cells type, e.g., renal cells, or cardiac cells. Method for stem cell differentiation into kidney and liver cells from adult bone marrow stem cells (BMSCs) are described for example by Forbes et al. (2002) Gene Ther 9:625-30. Protocols for the in vitro differentiation of embryonic stem cells into cells such as cardiomyocytes, representing all specialized cell types of the heart, such as atrial-like, ventricular-like, sinus nodal-like, and Purkinje-like cells, have been established (See e.g., Boheler et al. (2002) Circ Res 91:189-201). Multipotent stem cells from metanephric mesenchyme can generate at least three distinct cell types; glomerular, proximal and distal epithelia, i.e., differentiation into a single nephron segment (See e.g., Herzlinger et al. (1992) Development 114:565-72). Human and primate embryonic stem cells have been successfully differentiated in vitro into derivatives of all three germ layers, including beating cardiac muscle cells, smooth muscles, and insulin-producing cells, among others (Itskovitz-Eldor et al. Mol. Med. (2000) 5: 88-95; Schuldiner et al. Proc. Natl. Acad. Sci. USA (2000) 97: 11307-11312; Kaufman et al. Blood (1999) 94: (Suppl. 1, part 1 of 2) 34a.).

A cell derived from a source other than the subject to be treated also can be useful if protected from immune rejection using, for example, microencapsulation or immunosuppression. Useful microencapsulation membrane materials include alginate-ploy-L-lysine alginate and agarose (see, for example, Goosen, Fundamentals of Animal Cell Encapsulation and Immobilization, CRC Press, Boca Raton (1993); Tai & Sun, FASEB J. 7:1061 (1993); Liu et al., Hum. Gene Ther. 4:291 (1993); and Taniguchi et al., Transplant, Proc. 24:2977 (1992), each of which is incorporated herein by reference.

For treatment of a human subject, the cell can be a human cell, although a non-human mammalian cell also can be useful. In particular, a human fibroblast, muscle cell, glial cell, neuronal precursor cell or neuron can be transfected with an expression vector to express and secrete Gilatide or an analog, derivative, fragment, or mimetic thereof. A primarily fibroblast can be obtained, for example, from a skin biopsy of the subject to be treated and maintained under standard tissue culture conditions. A primary muscle cell can also be useful for transplantation. Considerations for neural transplantation are described, for example, in Chang, supra, 1995.

A cell derived from the central nervous system can be particularly useful for transplantation to the central nervous system since the survival of such a cell is enhanced within its natural environment. A neuronal precursor cell is particularly useful in the method of the invention since a neuronal precursor cell can be grown in culture, transfected with an expression vector and introduced into an individual, where it is integrated. The isolation of neuronal precursor cells, which are capable of proliferating and differentiating into neurons and glial cells, is described in Renfranz et al., Cell 66:713-729 (1991), which is incorporated herein by reference.

Methods of transfecting cells ex vivo are well known in the art (Kriegler, Gene Transfer and Expression: A Laboratory Manual, W. H. Freeman & Co., New York (1990)). For the transfection of a cell that continues to divide such as a fibroblast, muscle cell, glial cell or neuronal precursor cell, a retroviral vector is preferred. For the transfection of an expression vector into a postmitotic cell such as a neuron, a replication-defective herpes simplex virus type 1 (HSV-1) vector is useful (During et al., Soc. Neurosci. Abstr. 17:140 (1991); Sable et al., Soc Neurosci. Abstr. 17:570 (1991), each of which is incorporated herein by reference).

C. Retroviral Vectors

The invention provides methods of treatment and prophylaxis by administering to a subject an effective amount of a therapeutic, i.e., retroviral vector or peptide of the present invention. In one aspect, the therapeutic is substantially purified.

As is apparent to those skilled in the art in view of the teachings of this specification, an effective amount of viral vector which must be added can be empirically determined. Representative doses are detailed below. Administration can be effected in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician. One particularly useful formulation comprises recombinant AAV virions in combination with one or more dihydric alcohols, and optionally, a detergent, such as a sorbitan ester. See, for example, WO 00/32233.

More than one transgene can be expressed by the delivered recombinant virion. Alternatively, separate vectors, each expressing one or more different transgenes, can also be delivered to the CNS as described herein. Furthermore, it is also intended that the viral vectors delivered by the methods of the present invention be combined with other suitable compositions and therapies. For instance, Parkinson's disease can be treated by coadministering a recombinant AAV virion expressing Gilatide into the CNS (e.g., into the CA1 area of the hippocampus, caudate nucleus or putamen of the striatum) and additional agents, such as AADC, dopamine precursors (e.g., L-dopa), inhibitors of dopamine synthesis (e.g., carbidopa), inhibitors of dopamine catabolism (e.g., MaOB inhibitors), dopamine agonists or antagonists can be administered prior or subsequent to or simultaneously with the recombinant virion encoding Gilatide. For example, the gene encoding AADC can be coadministered to the CNS along with the gene encoding Gilatide. Similarly, L-dopa and, optionally, carbidopa, may be administered systemically. In this way, the dopamine which is naturally depleted in PD patients, is restored, apparently by AADC which is able to convert L-dopa into dopamine. Where the transgene is under the control of an inducible promoter, certain systemically delivered compounds such as munsterone, ponasteron, tetracyline or aufin may be administered in order to regulate expression of the transgene.

Recombinant AAV virions may be introduced into cells of the CNS using either in vivo or in vitro (also termed ex vivo) transduction techniques to treat preexisting neuronal damage. If transduced in vitro, the desired recipient cell will be removed from the subject, transduced with rAAV virions and reintroduced into the subject. Alternatively, syngeneic or xenogeneic cells can be used where those cells will not generate an inappropriate immune response in the subject. Additionally, neural progenitor cells can be transduced in vitro and then delivered to the CNS.

Suitable methods for the delivery and introduction of transduced cells into a subject have been described. For example, cells can be transduced in vitro by combining recombinant AAV virions with cells to be transduced in appropriate media, and those cells harboring the DNA of interest can be screened using conventional techniques such as Southern blots and/or PCR, or by using selectable markers. Transduced cells can then be formulated into pharmaceutical compositions, as described above, and the composition introduced into the subject by various techniques as described below, in one or more doses.

For in vivo delivery, the rAAV virions will be formulated into pharmaceutical compositions and one or more dosages may be administered directly in the indicated manner. A therapeutically effective dose will include on the order of from about 10⁶ to 10¹⁵ of the rAAV virions, more preferably 10⁷ to 10¹², and even more preferably about 10⁸ to 10¹⁰ of the rAAV virions (or viral genomes, also termed “vg”), or any value within these ranges. Generally, from 0.01 to 1 ml of composition will be delivered, preferably from 0.01 to about 0.5 ml, and preferably about 0.05 to about 0.3 ml, such as 0.08, 0.09, 0.1, 0.2, etc. and any integer within these ranges, of composition will be delivered.

Recombinant AAV virions or cells transduced in vitro may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J. Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). Cerebellar injections are complicated by the fact that stereotaxic coordinates cannot be used to precisely target the site of an injection; there is animal to animal variation in the size of cerebellar lobules, as well as their absolute three-dimensional orientation. Thus, cholera toxin subunit b (CTb) may be used to determine the exact location of the injection and reveal the pool of transducable neurons at an injection site. Injections may fill the molecular layer, Purkinje cell layer, granule cell layer and white matter of the arbor vitae but do not extend to the deep cerebellar nuclei.

One mode of administration to the CNS uses a convection-enhanced delivery (CED) system. In this way, recombinant virions can be delivered to many cells over large areas of the brain. Moreover, the delivered vectors efficiently express transgenes in CNS cells (e.g., neurons or glial cells). Any convection-enhanced delivery device may be appropriate for delivery of viral vectors. In a preferred embodiment, the device is an osmotic pump or an infusion pump. Both osmotic and infusion pumps are commercially available from a variety of suppliers, for example Alzet Corporation, Hamilton Corporation, Alza, Inc., Palo Alto, Calif.). Typically, a viral vector is delivered via CED devices as follows. A catheter, cannula or other injection device is inserted into CNS tissue in the chosen subject. One having ordinary skill in the art could readily determine which general area of the CNS is an appropriate target. For example, when delivering AAV-Gilatide to treat PD, the striatum is a suitable area of the brain to target. Stereotactic maps and positioning devices are available, for example from ASI Instruments, Warren, Mich. Positioning may also be conducted by using anatomical maps obtained by CT and/or MRI imaging of the subject's brain to help guide the injection device to the chosen target. Moreover, because the methods described herein can be practiced such that relatively large areas of the brain take up the viral vectors, fewer infusion cannula are needed. Since surgical complications are related to the number of penetrations, this mode of delivery serves to reduce the side effects seen with conventional delivery techniques. For a detailed description regarding CED delivery, see U.S. Pat. No. 6,309,634, incorporated herein by reference in its entirety.

D. Monitoring Treatment

Regeneration of neurons and hence treatment of disease may also be monitored by measuring specific neurotransmitters. For example dopamine levels can be monitored using known methods following administration of Gilatide and/or analogs. To measure dopamine content, a labeled tracer is administered to the subject. The detection of the label is indicative of dopamine activity. Preferably, the labeled tracer is one that can be viewed in vivo in the brain of a whole animal, for example, by positron emission tomograph (PET) scanning or other CNS imaging techniques. See, for example, U.S. Pat. No. 6,309,634 for methods of measuring dopamine content in vivo. By treatment of disease, as used herein, is meant the reduction or elimination of symptoms of the disease of interest, as well as the regeneration of neurons. Thus, dopamine levels prior and subsequent to treatment can be compared as a measure of neuron regeneration. Alternatively, visual symptoms of disease can be used as a measure of treatment. For example, memory tests can be monitored for improvement following treatment. Two commonly used tests to monitor dementia are the Wechsler Adult Intelligence Scale and the Cambridge Cognitive Test (CAMCOG). These tests have a number of different sections and test a variety of things, including the ability to learn new things and the ability to comprehend arithmetic and vocabulary.

Tissues can be harvested from treated subjects, and processed for evaluation of neuronal degeneration, regeneration and differentiation using routine procedures. In this invention it is useful to evaluate, for example, various cells of the striatum and substantia nigra (SN), such as examining coronal sections of the striatum and SN. Measurements performed over time can indicate increasing correction of cells distant to the vector administration site. Levels of dopamine and its metabolites, HVA and DOPAC, can be evaluated using high-performance liquid chromatography (HPLC) as described previously (Shen, Y., Hum. Gene Ther. (2000) 11:1509-1519). CSF can also be collected and evaluated for protein levels or enzyme activity, particularly if the vector encodes a secretable form of Gilatide peptide. Subjects can also be tested for rotational behavior periodically by intraperitoneal injection of apomorphine-HCl.

E. Pharmaceutical Compositions

The pharmaceutical compositions of the invention can be prepared in various manners well known in the pharmaceutical art. The carrier or excipient may be a solid, semisolid, or liquid material that can serve as a vehicle or medium for the active ingredient. Suitable carriers or excipients are well known in the art and include, but are not limited to saline, buffered saline, dextrose, water, glycerol, ethanol, and combinations thereof. The pharmaceutical compositions may be adapted for oral, inhalation, parenteral, or topical use and may be administered to the patient in the form of tablets, capsules, aerosols, inhalants, suppositories, solutions, suspensions, powders, syrups, and the like. As used herein, the term “pharmaceutical carrier” may encompass one or more excipients. In preparing formulations of the compounds of the invention, care should be taken to ensure bioavailability of an effective amount of the agent. Suitable pharmaceutical carriers and formulation techniques are found in standard texts, such as Remington's Pharmaceutical Sciences, Mack Publishing Co., Easton, Pa.

Compositions will comprise sufficient genetic material to produce a therapeutically effective amount of Gilatide peptide or analog, i.e., an amount sufficient to reduce or ameliorate symptoms of the disease state in question or an amount sufficient to confer the desired benefit. The compositions can contain a pharmaceutically acceptable carrier. Such carriers include any pharmaceutical agent that does not itself induce the production of antibodies harmful to the individual receiving the composition, and which may be administered without undue toxicity. Pharmaceutically acceptable carriers include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable carriers and excipients is available in Remington's Pharmaceutical Sciences (Mack Pub. Co., N.J. 1991).

For oral administration, the compounds can be formulated into solid or liquid preparations, with or without inert diluents or edible carrier(s), such as capsules, pills, tablets, troches, powders, solutions, suspensions or emulsions. The tablets, pills, capsules, troches and the like also may contain one or more of the following adjuvants: binders such as microcrystalline cellulose, gum tragacanth or gelatin; excipients such as starch or lactose; disintegrating agents such as alsinic acid, Primogel™, corn starch and the like; lubricants such as stearic acid, magnesium stearate or Sterotex™; glidants such as colloidal silicon dioxide; sweetening agents such as sucrose or saccharin; and flavoring agents such as peppermint, methyl salicylate or fruit flavoring. When the dosage unit form is a capsule, it also may contain a liquid carrier such as polyethylene glycol or fatty oil. Materials used should be pharmaceutically pure and non-toxic in the amounts use. These preparations should contain at least 0.05% by weight of the therapeutic agent, but may be varied depending upon the particular form and may conveniently be between 0.05% to about 90% of the weight of the unit. The amount of the therapeutic agent present in compositions is such that a unit dosage form suitable for administration will be obtained.

For the purpose of parenteral administration, the therapeutic agent may be incorporated into a solution or suspension. These preparation should contain at least 0.1% of the active ingredient, but may be varied to be between 0.1% and about 50% of the weight thereof. The amount of the active ingredient present in such compositions is such that a suitable dosage will be obtained.

The solutions or suspensions also may include one or more of the following adjuvants depending on the solubility and other properties of the therapeutic agent: sterile diluents such as water for injections, saline solution, fixed oils, polyethylene glycols, glycerine, propylene glycol or other synthetic solvents; antibacterial agents such as benzyl alcohol or methyl paraben; antioxidants such-as ascorbic acid or sodium bisulfite; chelating agents such as ethylene diaminetetraacetic acid; buffers such as acetates, citrates or phosphates; and agents for the adjustment of toxicity such as sodium chloride or dextrose. The parenteral preparation can be enclosed in ampules, disposable syringes or multiple dose vials made of glass or plastic.

The compounds can be administered in the form of a cutaneous patch, a depot injection, or implant preparation, which can be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers and synthetic silicones. Further information on suitable pharmaceutical carriers and formulation techniques are found in standard texts such as Remington's Pharmaceutical Sciences.

The exact amount of a therapeutic of the invention that will be effective in the treatment of a particular disease or disorder will depend on a number of factors that can be readily determined by the attending diagnostician, as one of ordinarily skilled in the art, by the use of conventional techniques and by observing results obtained under analogous circumstances. Factors significant in determining the dose include: the dose; the species, subject's size, age and general health; the specific disease involved, the degree of or involvement of the severity of the disease; the response of the individual patient; the particular compound administered; the mode of administration; the bioavailability characteristics of the preparation administered; the dose regimen selected; the use of concomitant medication; and other relevant circumstances specific to the subject. Effective doses optionally may be extrapolated from dose-response curves derived from in vitro or animal model test systems. In general terms, an effective amount of a peptide of the instant invention to be administered systemically on a daily basis is about 0.1 μg/kg to about 1000 μg/kg.

The invention also provides a pharmaceutical pack or kit comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

The preferred form depends on the intended mode of administration and therapeutic application. The preferred mode of administration is parenteral (e.g., intravenous, subcutaneous, intraperitoneal, intramuscular, intranasal). In a preferred embodiment, the pharmacological agent is administered by intranasally. The efficacy of intranasally administered Gilatide compared to GLP-1 can reflect differential entry into the CNS. GLP-1 penetrates the blood-brain-barrier following intravenous administration via simple diffusion (A. J. Kastin et al. J. Mol. Neurosci. 18, 7 (2002)), however Gilatide, containing just 9 amino acids and a stearic acid residue is likely to cross the nasal epithelium and enter the brain more efficiently than the 29 amino acid GLP-1.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., the pharmacological agent) in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The Gilatide peptide or analog of the present invention can be administered by a variety of methods known in the art. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. (See, e.g., Sustained and Controlled Release Drug Delivery Systems, J. R. Robinson, ed., Marcel Dekker, Inc., New York, 1978; U.S. Pat. No. 6,333,051 to Kabanov et al., and U.S. Pat. No. 6,387,406 to Kabanov et al.)

In certain embodiments, Gilatide peptide or analogs of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

In certain embodiments, a Gilatide peptide or analogs of the present invention can be administered in a liquid form. The pharmacological agent of the present invention is freely soluble in a variety of solvents, such as for example, methanol, ethanol, and isopropanol. The pharmacological agent is, however, highly lipophilic and, therefore, substantially insoluble in water. A variety of methods are known in the art to improve the solubility of the pharmacological agent in water and other aqueous solutions. For example, U.S. Pat. No. 6,008,192 to Al-Razzak et al. teaches a hydrophilic binary system comprising a hydrophilic phase and a surfactant, or mixture of surfactants, for improving the administration of lipophilic compounds such as the pharmacological agent of the present invention.

Supplementary active compounds can also be incorporated into the compositions. In certain embodiments, a Gilatide peptide or analog of the invention is coformulated with and/or coadministered with one or more additional therapeutic agents that are useful for improving the pharmacokinetics of the pharmacological agent. A variety of methods are known in the art to improve the pharmacokinetics of the pharmacological agent of the present invention. For example, U.S. Pat. No. 6,037,157 to Norbeck et al. discloses a method for improving the pharmacokinetics of the pharmacological agent by coadministration of the pharmacological agent and a drug that is metabolized by the cytochrome P450 monooxygenase, such as for example, the P450 3A4 isozyme.

Other methods of improving the pharmacokinetics of the Gilatide peptides or analogs have been disclosed, for example, in U.S. Pat. No. 6,342,250 to Masters, U.S. Pat. No. 6,333,051 to Kabanov et al., U.S. Pat. No. 6,395,300 to Straub et al., U.S. Pat. No. 6,387,406 to Kabanov et al., and U.S. Pat. No. 6,299,900 to Reed et al. Masters discloses a drug delivery device and method for the controlled release of pharmacologically active agents including the pharmacological agent of the present invention. The drug delivery device disclosed by Masters is a film comprising one or more biodegradable polymeric materials, one or more biocompatible solvents, and one or more pharmacologically active agents dispersed uniformed throughout the film. In U.S. Pat. No. 6,333,051, Kabanov et al. disclose a copolymer networking having at least one cross-linked polyamine polymer fragment, at least one nonionic water-soluble polymer fragment, and at least one suitable biological agent, including the pharmacological agent of the present invention. According to the teachings of this patent, this network, referred to as a nanogel network, improves the therapeutic effect of the pharmacological agent by decreasing side effects and increasing therapeutic action. In another patent, U.S. Pat. No. 6,387,406, Kabanov et al. also disclose another composition for improving the oral delivery of numerous pharmacological agents, including the pharmacological agent of the present invention. This delivery vehicle comprises a biological agent and a poly(oxyehtylene)-poly(oxypropylene) block copolymer. Straub et al. disclose porous drug matrices for use with drugs, and in particular, for use with low-aqueous solubility drugs, for enhancing solubility of the drug in an aqueous solution. Reed et al. disclose a drug delivery system, which uses a dermal penetration enhancer to transport a variety of physiologically active agents, including the pharmacological agent of the present invention, across a dermal surface or mucosal membrane of a subject.

Gilatide peptides or analogs of the present invention can be used alone or in combination to treat neurodegenerative disorders to produce a synergistic effect. Likewise, the pharmacological agent can be used alone or in combination with an additional agent, e.g., an agent which imparts a beneficial attribute to the therapeutic composition, e.g., an agent which effects the viscosity of the composition. The combination can also include more than one additional agent, e.g., two or three additional agents if the combination is such that the formed composition can perform its intended function.

The pharmaceutical compositions of the invention may include a “therapeutically effective amount” or a “prophylactically effective amount” of a pharmacological agent of the invention. A “therapeutically effective amount” refers to an amount effective, at dosages and for periods of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a Gilatide peptide of the invention is between 0.1 pg/kg to 1,000 mg/kg body weight, administered twice per day. Preferably, administration of a therapeutically effective amount of Gilatide peptide results in a concentration of pharmacological agent in the bloodstream that is between about 0.1 μM and 1000 μM. More preferably, the concentration of pharmacological agent in the blood is between about 0.1-100 μM. More preferably, the concentration of pharmacological agent in the blood is between about 0.1-10 μM. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### EXAMPLES

The following examples illustrate that the methods and compositions of the present invention can be employed to enhance cognition, learning and memory, induce insulin secretion, relieve CNS disorders, modulate memory disorders, and improve neuroprotective effects. The invention is demonstrated in the following examples in which art-recognized models are employed. The following examples are merely illustrative of the present invention and should not be construed so as to limit the scope of this invention.

### Example 1

### Materials and Methods

(i) Materials

Male Sprague Dawley rats (˜300 gm) housed under controlled lighting and ad libitum food were used for all studies. CD-1 wild-type GLP-1R mice were obtained from Charles River Laboratory. GLP-1R−/− mice were produced on a Charles River13 Laboratory CD-1 background as previously described (L. A. Scrocchi et al., Nat. Med. 2, 1254 (1996)). All mice were tested at 8 weeks of age.

(ii) Passive Avoidance Studies.

Passive avoidance was performed in an apparatus (MED Associates Inc., St. Albans, Vt.) consisting of one dark chamber and one light chamber that can be divided by a guillotine door. The training procedure was executed as previously described (N. Venable et al. Psychopharmacology 100, 215 (1990)). Rats were administered a 1.0 mA shock for 3 sec, mice a 0.5 mA shock for 5 sec. Retention tests were performed either at 1, 3 or 7 days post-pairing. Maximum latency was 600 sec for rats, 300 sec for mice. When pairing, if a rat or mouse did not enter the dark chamber within 2 min, the animal was excluded from the study.

(iii) Contextual Fear Conditioning.

Fear conditioning was performed in a modified apparatus (MED Associates Inc., St. Albans, Vt.) housed in a sound attenuated cubicle. A fan built into the cubicle also blocked any extraneous noise. The animal was placed in the chamber and the occurrence of freezing behavior measured every 10 sec for 2 min before a shock was administered (rats, 1.0 MA, 2 sec; mice, 0.5 mA, 5 sec). Freezing behavior was measured again (for 5 min) the next day. The apparatus was cleaned with 1% acetic acid following conditioning of each animal.

(iv) Morris Water Maze (MWM) Assessing Spatial Learning.

Spatial learning was assessed using the Morris Water Maze (Morris et al. Nature, 1982, 297: 681). Information was relayed via a tracker (HSV Image, Hampton, UK) to a personal computer, which was quantified by a specialized computer program (Water 2020, Hampton, UK). The acquisition study was performed as described previously. Animals were anaesthetized with isofluorane then administered peptide intra-nasally 20 min before testing. Rats were given four training trials. 48 h after training, a retention test was performed: rats were allowed to find the hidden platform for one trial. Mice initially had six trials without treatment to habituate them to the procedure. The following day, they were administered vehicle or [Ser(2)]exendin(1-9) (SEQ ID NO:1) and trained for an additional four trials with the platform in a new location. For a retention test, mice were given one trial to locate the platform the next day. Latency to find the platform was considered a measure of retention of spatial learning.

(v) Elevated Plus Maze.

The elevated plus maze consists of two open and two closed arms; time spent and the number of entries into open arms are indicators of neophobic anxiety in rats. The number of entries and total time spent in the open arms were tabulated over 5 min by an observer blind to the experimental condition. Animals falling off the apparatus were eliminated from the study. For the intracerebroventricular (i.c.v.) studies, rats were implanted with a cannula (22 gauge, Plastics One, Roanoke, Va.) into the left ventricle (AP 0.8 mm, ML 1.6 mm, DV 3.5 mm from dura) then allowed at least 3-4 days to recover. The peptides were infused in a total volume of 2 μl (1 μl min −1) 25 min before training with two trials per day for 5 days. The visual platform test was conducted after the last training trial on day 5 in a different location of the pool.

(vi) Cloning into rAAV Vector

A 1.4 kb rat GLP-1 receptor cDNA was cloned into an rAAV backbone containing the 1.1 kb CMV enhancer/chicken β-actin CBA promoter, 800 bp human interferon-β scaffold attachment region (SAR) inserted 5′ of the promoter, the woodchuck post-transcriptional regulatory element (WPRE) and the bovine growth hormone polyA (rAAVGLP-1R). EGFP (Clontech, Palo Alto, Calif.) was inserted into the same rAAVSAR-CBA-WPRE-bGH backbone (rAAVEGFP). EGFP uses the enhanced green fluorescent protein (EGFP) as a transformation marker. EGFP is a red-shifted variant of wild-type GFP from Aquorea victoria. AAV1/AAV2 pseudotype vectors (virions containing a 1:1 ratio of AAV1 and AAV2 capsid proteins with AAV2 ITRs) were generated. HEK 293 cells were transfected with the AAV cis plasmid, the AAV1 and AAV2 helper plasmids and the adenovirus helper plasmid by the standard calcium phosphate method. 48 hr after transfection, the cells were harvested and the virus purified using heparin affinity columns (Sigma, St Louis, Mo.) according to the method of Clark et al. (K. R. Clark et al. Hum. Gene Ther. 1999, 10:1031-1039). The genomic titer of each virus was determined quantitatively using the ABI 7700 real time PCR machine (Applied Biosystems, Foster City, Calif.) with primers designed to WPRE. Adult male Sprague Dawley rats (250·300 gm, Charles River) were injected with either rAAVEGFP or rAAVGLP-1R vector (3×10 915 particles) in 2 μl plus 1 μl of 20% mannitol bilaterally into the dorsal hippocampus (±3.8 mm AP, ±1.8 mm ML, ±3.4 mm DV from skull). Vectors were infused at a rate of 200 nl min⁻¹ using a microprocessor-controlled mini-pump. rAAVGilatide or rAAVGilatide analogs can be made following the above protocol.

(vii) In Situ Hybridization

Three weeks after injection of rAAV vectors, brains were removed and immediately frozen on dry ice. Cryostat-cut 16 μm coronal sections were collected on poly-L-lysine coated slides before post-fixing. After dehydration in 100% ethanol, slides were air-dried then 50 μl hybridization buffer containing 5×10 5 cpm 35 S-labeled probe was applied to each section. Slides were incubated overnight at 37° C. then washed 2× at 55° C. for 15 min in 1×SSC, 10 mM DTT, then 2×15 min in 0.5×SSC, 1 mM DTT. Sections were dehydrated, air dried, and exposed to Kodak Biomax MR-1 film (Amersham Biosciences, Piscataway, N.J.) for 6 days. WPRE-AS probe sequence was 5′AGC ATG ATA CAA AGG CAT TAA AGC AGC GTA TCC ACA TAG 3′. The probe was labeled by combining 5 pmol oligo, 3 μl TdT buffer, 25 μCi [35 S]•-thio-dATP (New England Nuclear, Boston, Mass.), 2 μl TdT (Life Technologies, Rockville, Mass.) with water to 15 μl and incubated at 37° C. Unlabeled probe was removed with a G-50 microcolumn (Amersham Biosciences).

### Example 2

### Gilatide Peptides Induce Insulin Production

To confirm biologic activity of Gilatide peptides or analogs, a rat insulinoma cell line expressing the GLP-1R (RINm5f) can be cultured and incubated the Gilatide peptide or analogs followed by an ELISA study as described below.

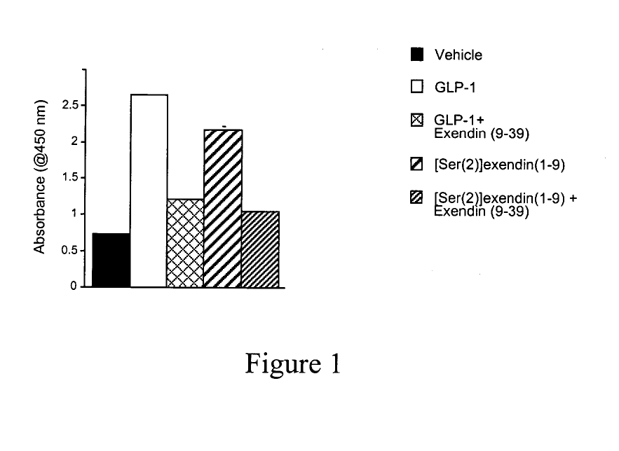

To confirm biological activity of synthesized Gilatide peptide ([Ser(2)]exendin(1-9)) that was synthesized with a stearic acid residue added to the N-terminus, GLP-1 or [Ser(2)]exendin(1-9) (10 nM) were incubated with the cultured rat insulinoma cell line expressing the GLP-1R (RINm5f) in the presence or absence of the GLP-1R antagonist, exendin(9-39) (10 nM). Rat insulinoma cells (RINm5f) were cultured in 24 well plates and incubated in serum-free medium for 1 hour before treatment. The GLP-1 peptide antagonist exendin (9-39) (10 nM) was added 1 hour prior to GLP-1 (7-36) or [Ser(2)]exendin(1-9) (10 nM). Cells were then incubated for 6 hours in the presence of either GLP-1 or [Ser(2)]exendin(1-9). Media were then removed and assayed for insulin concentrations via ELISA (Pennisula Labs, San Carlos, Calif.). ELISA of the culture media for insulin showed that both GLP-1 (7-36) and [Ser(2)] exendin (1-9) stimulated insulin release (P<0.001) was blocked by exendin (9-39) (FIG. 1).

### Example 3

### Gilatide Increases Passive Avoidance Response

In the instant invention, rats were pretreated intranasally with one of three dose levels (10 μg/kg, 30 μg/kg, or 60 μg/kg) of Gilatide in 5% β cyclodextrin or an octamer having a sequence homology to CRH and urocortin. The native forms of these latter peptides previously have been shown to have some potential efficacy in memory facilitation. A control group received vehicle (5% cyclodextrin) alone. With three dose levels for each of the peptides studied, a total of seven (7) groups were employed, each group having 5-8 rats, for a total of 50 rats tested. On the first day of conditioning, the pretreated rats (N=7-13) were administered a single foot shock trial (0.1 mA over 3 seconds) after entering the dark compartment. The animals were replaced in the test apparatus and latencies again were measured on Days 1, 3, 7, and 21 following the aversive stimulus.

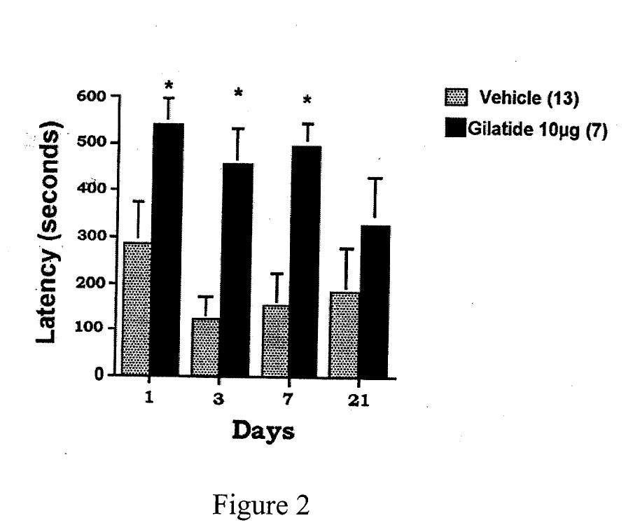

As predicted, the control animals (N=13) showed short latencies to enter the dark room (mean±SEM=15.4±3.8) prior to exposure to the single mild shock. Similarly, all other groups had increased latencies ranging from 14.8 to 31.6 seconds. At 24 hours (Day 1) following the initial test, and delivery of the single shock, the animals were replaced in the test apparatus and latency again measured. Those control rats, which had learned that the aversive stimulation was associated with entering the dark room, had mean latencies of 286.3±88.8 seconds. (FIG. 2) Similarly, all other groups had increased latencies, ranging from 342.5 to 542.9 seconds. Those rats (N=7) that received 10 μg of Gilatide had a mean latency of 542.9 seconds, an increase in latency of 90% above those rats administered vehicle alone. This difference was statistically significant (p<0.05).

On Day 3, rats were again tested in the apparatus. By this time the control rats had started to forget the aversive stimulus; thus, their latencies decreased to 125.6±51.4 seconds. (FIG. 2) Similarly, all other groups, except one, had a drop in latencies, with values ranging from 118.4 to 279 seconds. Of interest, the rats administered 10 μg Gilatide maintained a mean latency of 458 seconds. This result was statistically significant at p=0.003 compared to the rats administered vehicle only. (FIG. 2)

On Day 7 following delivery of the peptide, the rats were again placed in the test apparatus. The rats administered 10 μg Gilatide had a mean latency of 501.1 seconds compared to the control (vehicle only) group, which had a mean latency of 157.6 (p=0.002). (FIG. 2) Finally, the effect was tested 21 days after the single episode of training. By this time, the memory facilitation was lost, although a trend was apparent even at this markedly delayed time point. (FIG. 2)

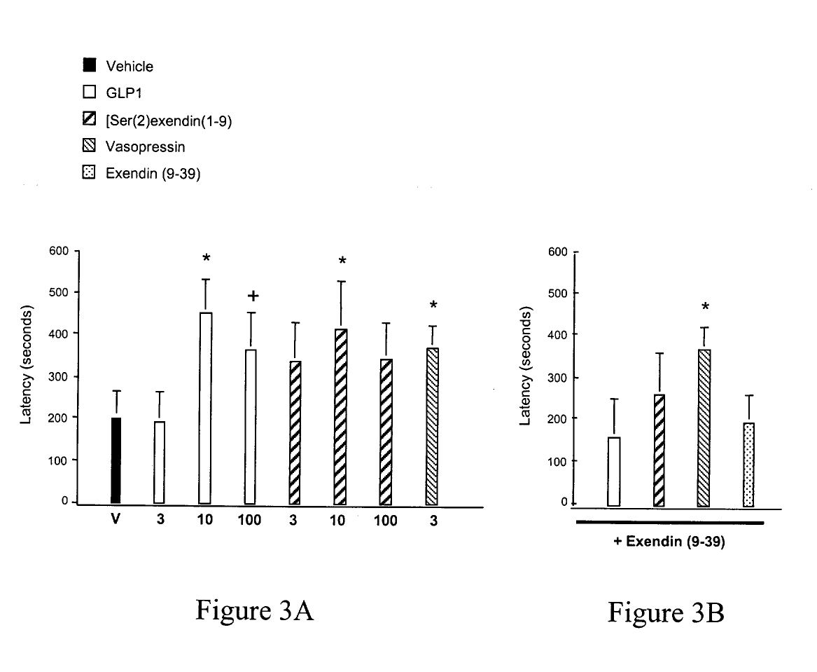

Gilatide peptides administered intracerebroventricularly (i.c.v.) also enhanced the passive avoidance. GLP-1 and [Ser(2)]exendin(1-9) administered intracerebroventricularly (i.c.v.) enhanced latency in the PA task, the effect being similar to that of vasopressin (FIG. 3A), a peptide previously shown to facilitate learning (DeWied, D. Nature, 1971, 232:58). FIG. x2A shows that GLP-1 (10 ng *P<0.05, 100 ng+P=0.01) and [Ser(2)]exendin(1-9) (10 ng *P<0.05) enhanced latency in PA similar to vasopressin (*P<0.05). Consistent with its action as a GLP-1R antagonist, co-infusion of exendin(9-39) completely blocked the memory enhancing effects of GLP-1 and [Ser(2)]exendin(1-9) but not vasopressin (FIG. 3B). GLP-1 and [Ser(2)]exendin(1-9) (100 ng i.c.v.) decrease latency (not shown) (GLP-1, Two-way ANOVA, F=13.42(1,80); P=0.01; [Ser(2)]exendin(1-9), F=5.08(1,80); P=0.02).

### Example 4

### Gilatide Peptides Enhances Spatial Learning: Morris Water Maze Studies

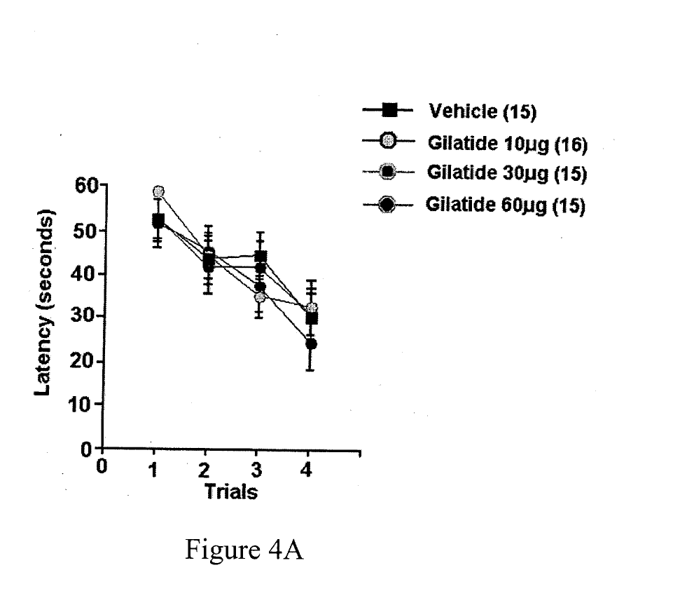

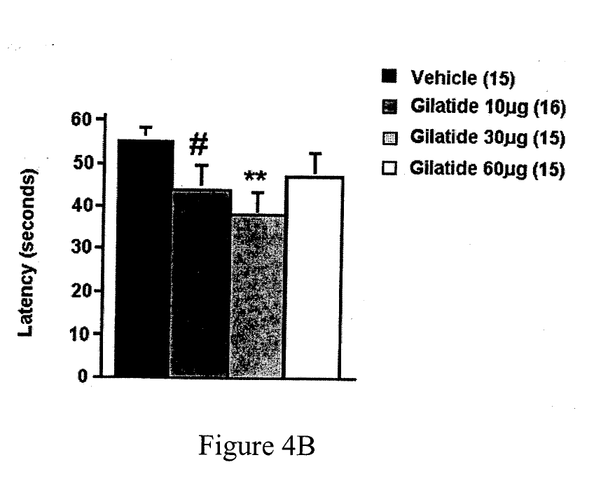

In another series of experiments, rats (N=15-16) were pretreated with either Gilatide (10 μg/kg, 30 μg/kg, or 60 μg/kg) or vehicle and then trained for four trials in a Morris Water Maze. Two days following training, the rats were retested. Latency to find a submerged platform in the Morris Water Maze paradigm was measured. There was no difference in acquisition between groups during training. (FIG. 4A) Retention tests 48 hours following training yielded a trend for significance at the Gilatide 10 μg dose (t=1.774(27); P=0.08) and significant difference between Gilatide 30 μg dose (t=2.76(26); P+0.01) compared to control (vehicle only (VEH)) (FIG. 4B).

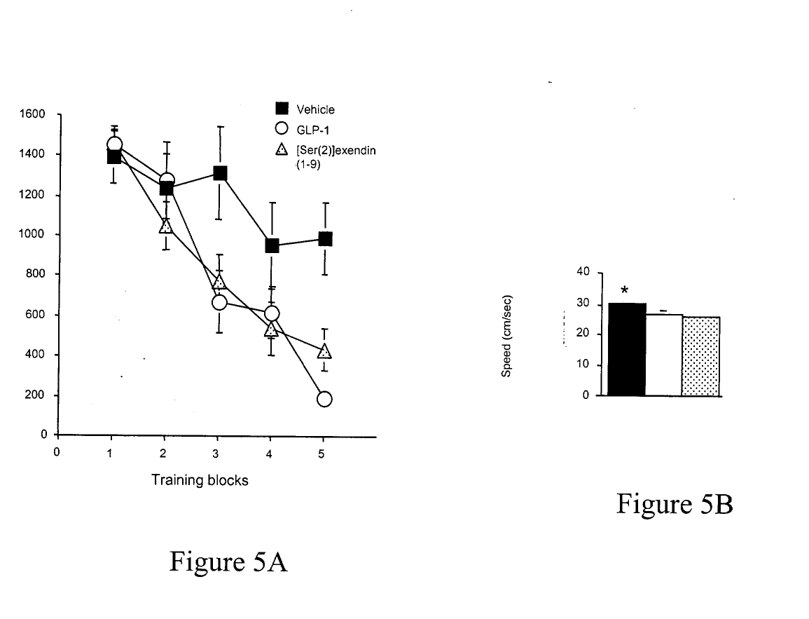

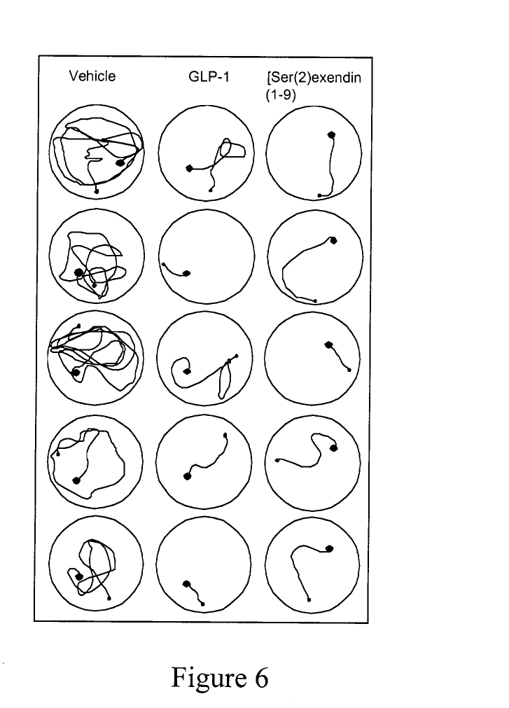

Assessment of the effects of i.c.v. GLP-1 and Gilatide peptide, [Ser(2)]exendin(1-9), on spatial learning in the MWM showed that both peptides significantly reduced distance traveled (FIG. 2C) to locate the platform compared to control rats. FIG. 5 shows the distance traveled (GLP-1, F=10.53 (1,80); P<0.01; [Ser(2)]exendin(1-9), F=7.28(1,80); P<0.01) to find a hidden platform in the MWM. Control rats swam faster than either GLP-1 or [Ser(2)]exendin(1-9) treated rats ruling out extraneous motor effects. FIG. 5B shows that both peptides decrease swimming speed compared to vehicle (P<0.05).

Furthermore, enhancement of associative and spatial learning by both peptides was not due to stress effects (see Table 4) or altered nociception (data not shown). FIG. 6 shows representative swimming path tracings of five individual rats on day 5 in the MWM. Close examination of individual rat search patterns on day 5 of training showed that although GLP-1 and [Ser(2)]exendin(1-9)-treated rats swam more slowly, they displayed a highly efficient search strategy compared to control rats (FIG. 6), suggestive of enhanced spatial learning.

### Example 5

### Route of Administration Comparison

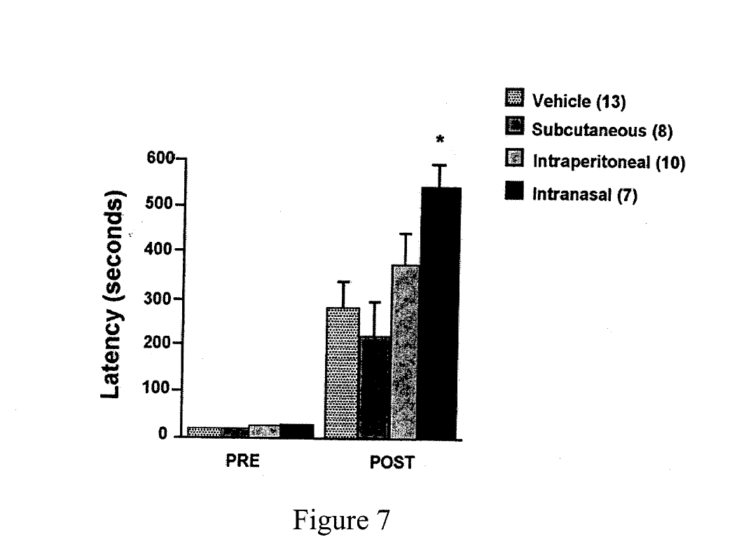

Rats were pretreated with either 33 10 μg/kg Gilatide in 5% β cyclodextrin or vehicle by one of three routes of administration: intranasally, subcutaneously, or intraperitoneally. On Day 0, the rats (N=7-13) were conditioned by administration of a single foot shock trial (0.1 mA over 3 seconds) after entry into the dark compartment of a passive avoidance apparatus (the same passive avoidance chamber used in the first series of experiments). At 24 hours (Day 1) following the initial test, and delivery of the single shock, the animals were replaced in the test apparatus and latency again measured. (FIG. 7)

Since the lowest dose of Gilatide tested, 10 μg, was effective, smaller doses were tested to determine the activity of smaller doses in this animal model. Rates (N=5-10) were pretreated intranasally with one of five dose levels (0.1 μg/kg, 1 μg/kg, 3 μg/kg, 30 μg/kg or 60 μg/kg) of Gilatide in 5% β cyclodextrin, vehicle (5% cyclodextrin), or Nicotine (0.3 mg/kg, subcutaneously). On Day 0, the rats were conditioned by administration of a single foot shock trial (0.1 mA over 3 seconds) after entry into the dark compartment of a passive avoidance apparatus (the same passive avoidance chamber used in the other experiments). The preconditioned rats were retested on Days 1, 3, 7, and 21.

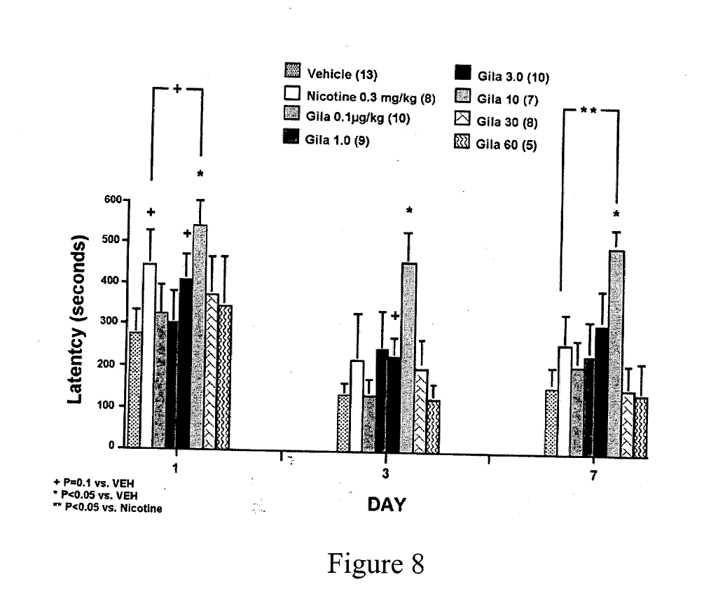

Although the rats administered either 0.1 or 1.0 μg/kg showed no effect, the rats receiving 3.0 μg/kg of Gilatide exhibited extended latencies at 3 and 7 days post conditioning. (FIG. 8) This trend was observed, but the effect did not reach statistical significance. The positive control group (0.3 mg/kg nicotine; the gold standard for this assay and a well-established nicotine dose in this task) exhibited modestly increased latencies at 24 hours. (FIG. 8) This effect, however, was transient and not as significant as the effect of Gilatide administered at 10 μg/kg. The effect was further tested at 21 days post the single episode training. By this time, however, the memory facilitation was lost, although there was a trend even at this markedly delayed time point.

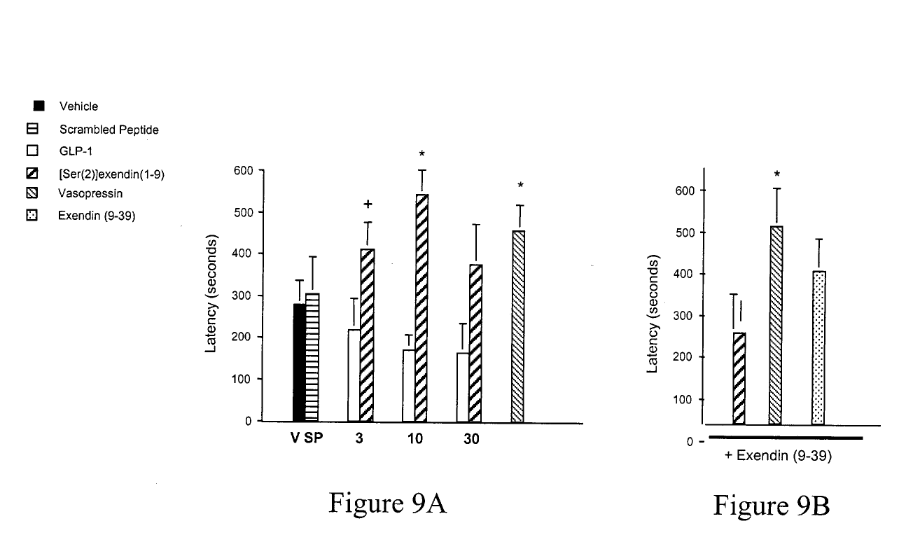

Central administration of drugs poses major problems for translation to clinical applications. We therefore investigated the potential for systemic administration, in particular, nasal delivery (Born et al. Nat Neurosci. 2002 5(6):514-516). Intranasal administration of [Ser(2)]exendin(1-9) but not GLP-1, increased latency in the PA test to a similar extent as vasopressin (FIG. 9A). A scrambled peptide, containing the same 9 amino acids as [Ser(2)]exendin(1-9), but in random order and not homologous to any known protein produced similar latency as vehicle. Co-administration of exendin (9-39) blocked the cognitive enhancing effects of [Ser(2)]exendin(1-9) but not vasopressin (FIG. 9B).

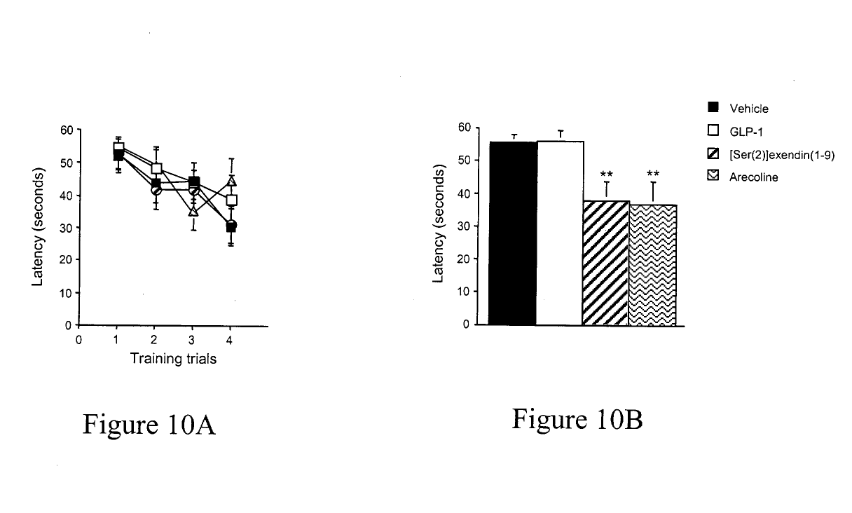

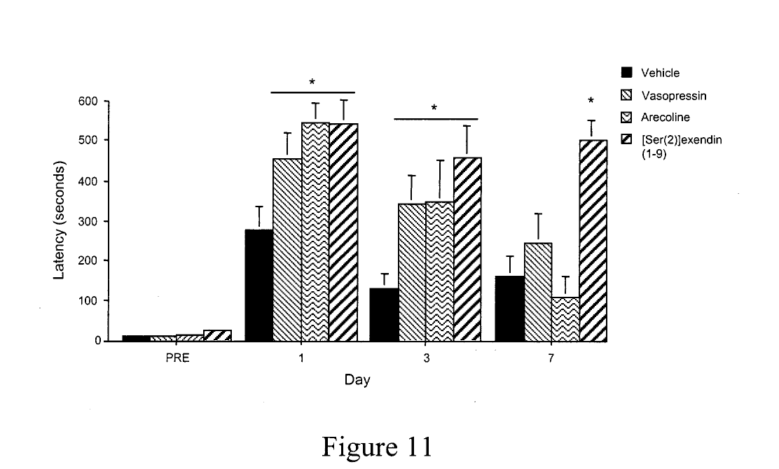

Clinically approved treatments for cognitive impairment act primarily on the cholinergic system. The effects of intranasal GLP-1 and [Ser(2)]exendin(1-9) were compared with that of the cholinergic agonist arecoline on spatial learning in a modified version of the MWM (Setlow, B. et al. Learn. Mem, 2000. 7: 187). Rats were first administered vehicle, [Ser(2)]exendin(1-9), GLP-1 or arecoline, and trained for four trials to locate a submerged platform. They were then tested in a single retention trial 48 hours after initial training. There were no differences between treatments in acquisition (FIG. 10A). In contrast, [Ser(2)]exendin(1-9) and arecoline, but not GLP-1, significantly reduced the latency for rats to locate the submerged platform in the retention trial (FIG. 10B).

In light of the strong effects of [Ser(2)]exendin(1-9) on retention in the MWM, multiple tests of retention were conducted using the PA paradigm to compare single pretreatment doses of intranasal [Ser(2)]exendin(1-9), vasopressin and arecoline. All 5 compounds produced similar latency times at 1 and 3 days following the initial pairing (FIG. 11). However, at 7 days post-pairing, [Ser(2)]exendin(1-9) was associated with significantly greater retention than vasopressin and arecoline. Together with the results from the modified MWM procedure, these data show that [Ser(2)]exendin(1-9) possesses robust effects on memory retention.

### Example 6

### Gilatide Effect on Fasting Blood Glucose Levels Depends on Route of Administration

Gilatide administered intraperitoneally (IP), but not intranasally (IN), lowers blood glucose levels in rats fasted for 24 hours. Table 2 shows that Gilatide IP administration lowers blood glucose. Gilatide's effect on blood glucose in fasting rats was measured. Groups of 24 hour fasting rats (n=5 or 6) were delivered either vehicle, insulin, Gilatide, GLP-1, exendin-4, Insulin A-chain, Insulin B-chain or C-peptide. Glucose levels were measured 20 minutes following intraperitoneal administration. All peptides were given at a dose of 100 ug (Table 2). In this experiment, insulin, the positive control, led to a 44% drop in blood glucose. Gilatide led to a significant drop in glucose level by 14%. However, none of the other active GLP-1/Exendin-4 peptides (nor the inactive insulin fragments) showed any efficacy at this does.

TABLE 2
Gilatide IP administration lowers blood glucose level

| | Blood glucose (mg/dl) |
|---|---|
| Group | mean ± s.e.m. |
| vehicle | 91 ± 3.6 |
| Insulin A chain | 90.3 ± 4.7 |
| Insulin B chain | 100 ± 4.9 |
| C peptide | 90.3 ± 8.9 |
| Insulin | 50.7 ± 8.2** (P < 0.01) |
| GLP1 | 89.5 ± 1.1 |
| Exendin-4 | 94.2 ± 2.2 |
| Gilatide | 78.5 ± 3.4* (P < 0.05) |

In contrast, intranasal GLP-1 lowered fasting blood glucose levels whereas [Ser(2)]exendin(1-9) did not (see Table 3). Disrupted glucose regulation, particularly hypoglycemia, is associated with impaired learning (Santucci A. et al. Behav. Neural. Biol., 1990, 53: 321). Intranasal GLP-1 is anxiogenic as shown by significantly increasing time spent in the closed arms of the elevated plus maze (Table 4). Therefore, the anxiogenic and hypoglycemic effects of intranasal GLP-1 may have compromised learning in both the PA and MWM paradigms.

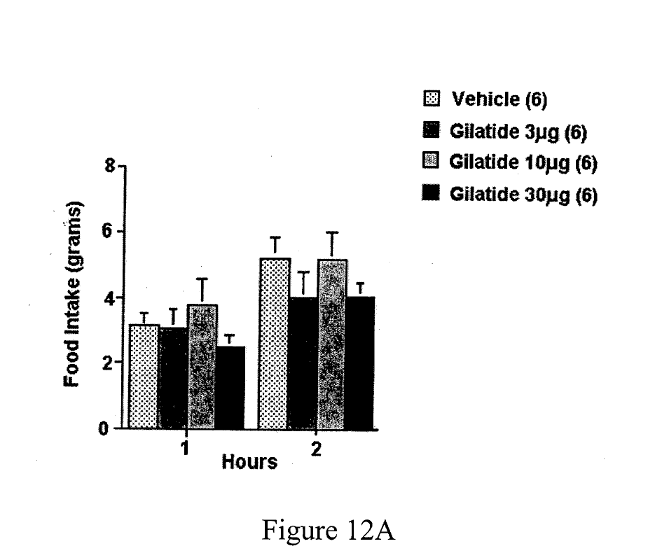

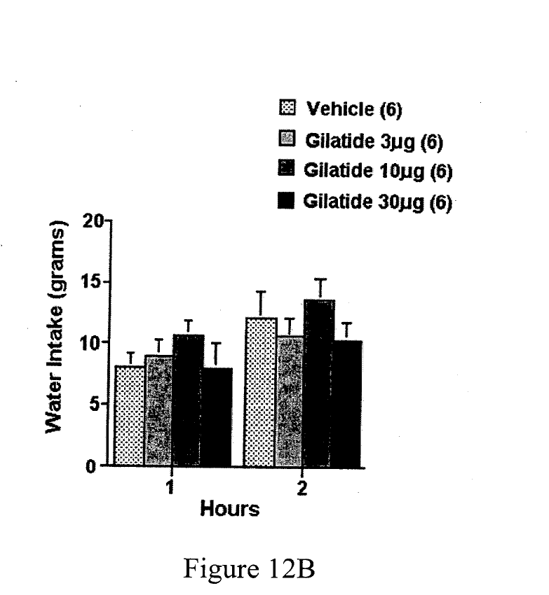

The effect of Gilatide administration was further tested by measuring the intake of food and water in rats following 18 hours of deprivation. Rats (N=6) were administered either one of three dose levels of Gilatide (3 μg/kg, 10 μg/kg, or 30 μg/kg) or vehicle and then deprived of food and water for 18 hours. Following deprivation, the rats were given access to food and water, and their intake levels of each were measured (FIGS. 12A and B). There were no significant differences between groups treated with Gilatide compared to vehicle.

TABLE 3
Effects of intra-nasal GLP-1 and [Ser(2)exendin(1-9)
on blood glucose (mg/dl).

| | | | [Ser(2)]exendin |
|---|---|---|---|
| Dose | Vehicle | GLP-1 | (1-9) |
| 3 μg | 84 ± 3.1 | 73.8 ± 2.2* P < 0.05 | 82.6 ± 4.5 |
| 10 μg | | 70.6 ± 2.5** P < 0.01 | 78.0 ± 3.2 |
| 30 μg | | 71.2 ± 2.1* P < 0.05 | 83.6 ± 2.8 |

TABLE 4
Anxiogenic effects of various treatments (intracerebroventricular (ICV) intraperitoneal
(IP) and intranasal (IN)) assessed with elevated plus maze test.

| | TIME | | ENTRIES | | |
|---|---|---|---|---|---|
| | Open Arm | Closed Arm | Open Arm | Closed Arm | |
| Vehicle (10) | 39.7 ± 7.9 | 131.8 ± 13.16 | 1.9 ± 0.3 | 3.8 ± 0.2 | IP |
| PTZ 20 mg/kg (9) | 0*** | 249.6 ± 13.32*** | 0*** | 1.6 ± 0.4 | IP |
| Midazolam | 141.3 ± 18*** | 89.2 ± 15 | 6.6 ± 0.7*** | 5.1 ± 0.5 | IP |
| 1.5 mg/kg (14) | | | | | |
| [Ser(2)]exendin | 27.7 ± 13 | 141.9 ± 20 | 1.4 ± 0.3 | 3.6 ± 0.6 | IN |
| (1-9) 10 μg (10) | | | | | |
| [Ser(2)]exendin | 33.1 ± 11 | 102 ± 13 | 1.8 ± 0.4 | 3.8 ± 0.6 | IN |
| (1-9) 30 μg (10) | | | | | |
| GLP-1 (7-36) | 11.6 ± 4* | 171.8 ± 10⁺ | 1.11 ± 0.3 | 4.8 ± 1.0 | IN |
| 10 μg (9) | | | | | |
| GLP-1 (7-36) | 22.1 ± 10 | 197.2 ± 18** | 1.33 ± 0.4 | 2.1 ± 0.4 | IN |
| 30 μg (9) | | | | | |
| Vehicle (7) | 20.4 ± 9 | 208.6 ± 19 | 1.5 ± 0.6 | 3.7 ± 0.7 | ICV |
| [Ser(2)]exendin | 16.7 ± 10 | 204.9 ± 19 | 1.4 ± 0.4 | 2.4 ± 0.6 | ICV |
| (1-9) 10 ng (7) | | | | | |
| GLP-1 (7-36) | 16.7 ± 11 | 198.3 ± 29 | 0.7 ± 0.4 | 2.7 ± 0.5 | ICV |
| 10 ng (7) | | | | | |
| EGFP over- | 15.7 ± 6.9 | 96.3 ± 35 | 0.88 ± 0.3 | 1.7 ± 0.3 | |
| expressers (9) | | | | | |
| GLP-1 over- | 33.2 ± 12 | 142.2 ± 23 | 1.5 ± 0.5 | 2.6 ± 0.7 | |
| expressers (11) | | | | | |
| GLP-1 +/+ (10) | 53.4 ± 15 | 177 ± 16 | 6 ± 1.7 | 5.7 ± 1.0 | |
| GLP-1 −/− (10) | 58.0 ± 13 | 142 ± 21 | 3.7 ± 0.8 | 4.9 ± 0.8 | |

### Example 7

### Gilatide's Effect on Memory Consolidation

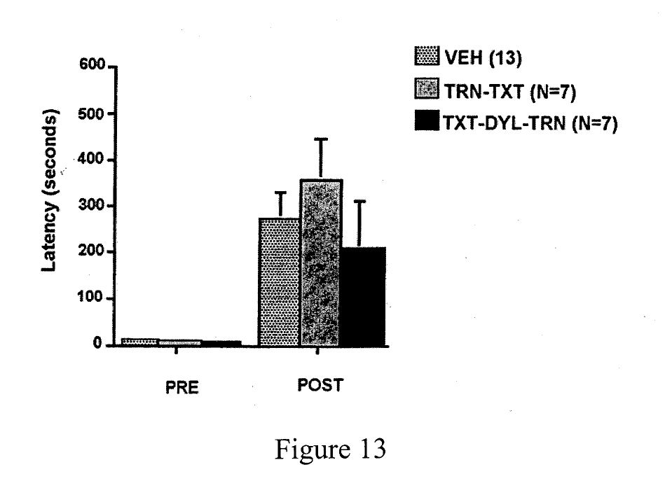

The effect of Gilatide was tested on memory consolidation by administering the peptide after shock testing. Rats (N=7-13) were preconditioned by administering a single foot shock trial (0.1 mA over 3 seconds) after entering the dark compartment of a passive avoidance apparatus. Twenty (20) minutes after the conditioning session, one group of rats was administered 10 μg/kg of Gilatide intranasally (TRN-TXT). Another group of rats (TXT-DLY-TRN) was administered this same dose of Gilatide 24 hours after the conditioning session. Both treatment groups were returned to the test apparatus 24 hours following treatment and latencies were again measured. There was no difference in latencies between the groups (p>0.05). (FIG. 13)

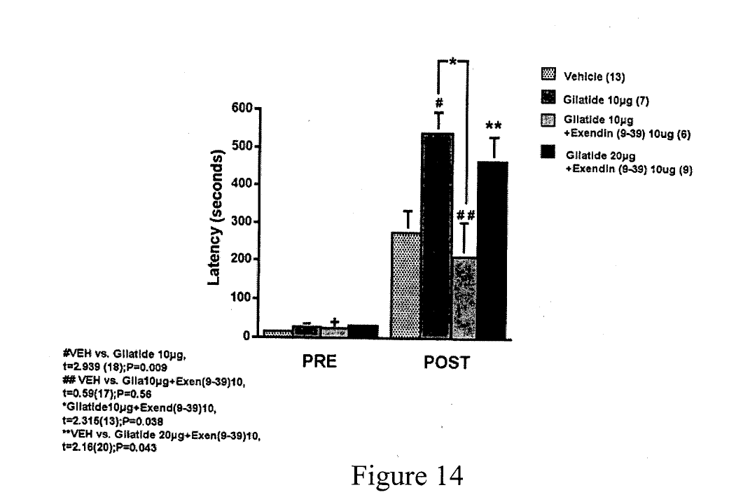

The effects of Gilatide when used with or without an Exendin-4 antagonist were observed and measured. Rats (N=6-13) were pretreated with either 10 μg/kg or 20 μg/kg of Gilatide with or without an Exendin-4 antagonist (10 μg/kg). A control group was administered vehicle alone. The pretreated rats were conditioned on Day 0 by administration of a single foot shock trial (0.1 mA over 3 seconds) after entry into the dark compartment of a passive avoidance apparatus (the same passive avoidance chamber used in the other experiments). The preconditioned rats were retested on 24 hours later. Co-treatment of Gilatide 10 μg/kg with an Exendin-4 antagonist (10 μg/kg) completely blocked enhancement of associative learning by Gilatide. (FIG. 14) Increasing the dose of Gilatide to 20 μg/kg surmounted the antagonism. (FIG. 14)

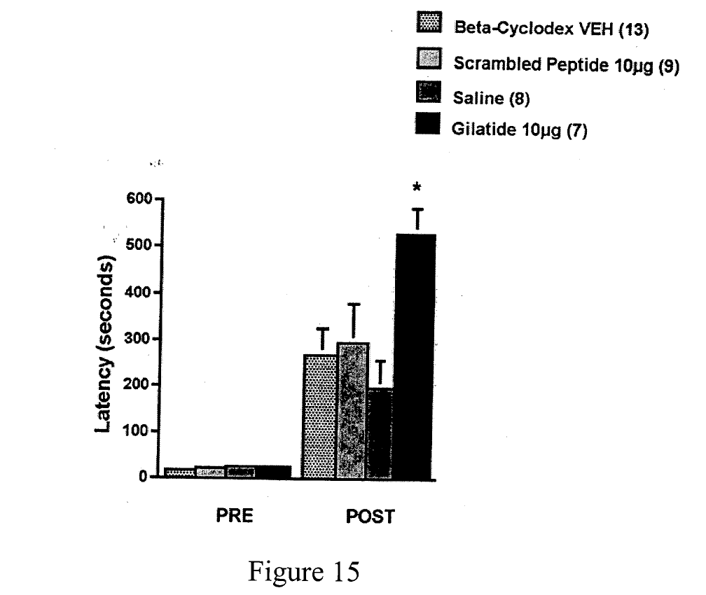

To further illustrate Gilatide's effect on passive learning in rats, rats (N=7-13) were pretreated with either Gilatide (10 μg/kg), saline (5 μl normal saline), a scrambled peptide (not matched to any active peptide) containing the same residues as Gilatide, or vehicle (5% β cyclodextrin) and conditioned on Day 0 by administration of a single foot shock trial (0.1 mA over 3 seconds) after entry into the dark compartment of a passive avoidance apparatus (the same passive avoidance chamber used in the other experiments). Twenty-four hours later the rats were returned to the apparatus and retested. The mean latencies of the groups of rats administered saline and the scrambled peptide did not differ from that of the control group (vehicle alone). (FIG. 15) In comparison, the rats administered Gilatide demonstrated a marked effect. (FIG. 15)

### Example 8

### Gilatide's Effect on Memory and Spatial Learning is Significantly Decreased in GLP-1 Receptor Knockout Mice (GLP-1R−/−)

These studies demonstrate that gilatide peptides and analogs interact with the GLP-1 receptor. In GLP-1R knockout mice, Gilatide's effect on memory and spatial learning is significantly decreased.

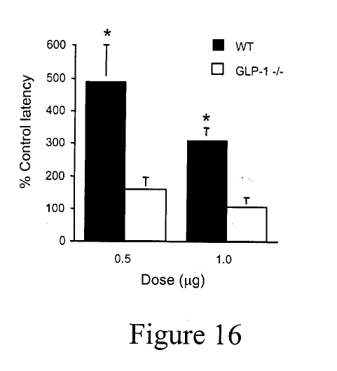

To further determine the specificity of the effects of [Ser(2)]exendin(1-9) on memory in vivo, parallel experiments were conducted in GLP-1R deficient (GLP-1R−/−) mice. Although such mice have mild fasting hyperglycemia and abnormal neuroendocrine responses, they have completely normal feeding behavior, fertility and general activity (Scrocchi et al. Nat. Med. 1996, 2:1254). Consistent with mediation of the memory enhancing effects of [Ser(2)]exendin(1-9) via GLP-1R, intranasal [Ser(2)]exendin(1-9) did not enhance associative learning in the knockout mice but did in wild-type GLP-1R+/+ mice (FIG. 16).

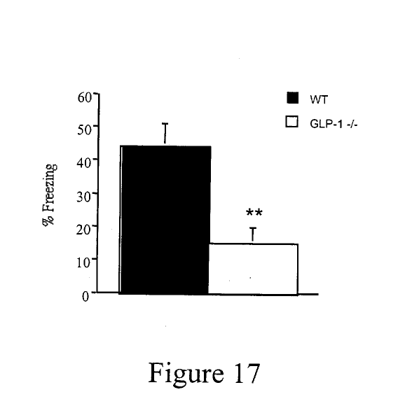

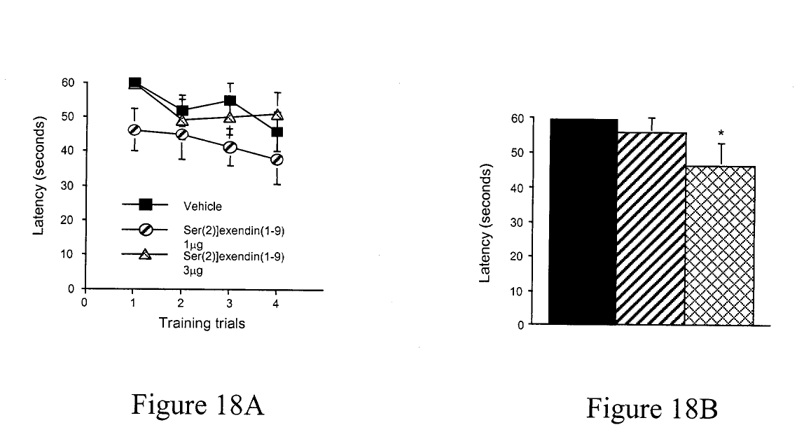

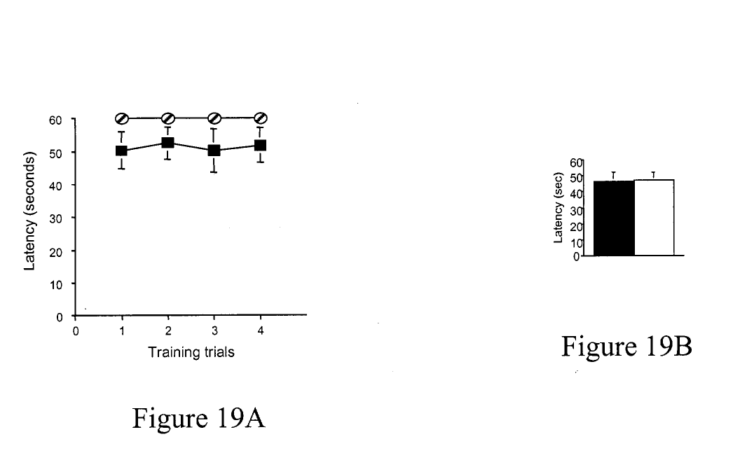

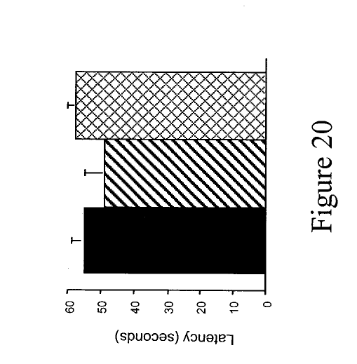

GLP-1R−/− mice were also tested in an associative learning paradigm: contextual fear conditioning. Mice were placed in a chamber and monitored for freezing behavior then administered a mild shock. The next day, they were placed in the same chamber and freezing behavior was again measured. Compared to GLP-1R+/+ mice, GLP-1R−/− mice demonstrated a marked decrease in contextual fear conditioning (FIG. 17). Again, differences in contextual learning between strains were likely not due to stress effects (Table 4).

[Ser(2)]exendin(1-9) tended to improve acquisition of spatial learning in GLP-1R+/+ mice (FIG. 18A) and significantly enhanced retention when tested 24 hours later (FIG. 18B). In contrast, GLP-1R−/− mice did not learn during the acquisition portion of the modified version of the MWM, and did not improve their performance following [Ser(2)]exendin(1-9) administration (FIG. 19A). Moreover, [Ser(2)]exendin(1-9) did not enhance retention of spatial learning in the GLP-1R−/− mice (FIG. 20). The differences in learning were not due to compromised visual acuity or locomotion, since latency to find a visual platform was the same for both strains of mice (FIG. 19B).

### Example 9

### Regulation of GLP-1R Expression by [Ser(2)]exendin(1-9) and Targeted Hippocampal Overexpression of GLP-1R in Rats.

To further investigate the putative central role of the GLP-1R in learning and memory, two groups of rats were tested in the PA paradigm: pre-treatment with either [Ser(2)]exendin(1-9) or vehicle. A third group was sham trained (shocked only). Immediately after pairing, the hippocampus of each rat was processed and real-time quantitative RT-PCR was used to detect changes in GLP-1R mRNA. Training (vehicle pre-treatment) produced an increase in GLP-1R mRNA compared with sham-shocked controls, while pre-treatment with intranasal [Ser(2)]exendin(1-9) decreased GLP-1R mRNA to the levels found in sham-shocked animals, and also significantly lowered the mRNA transcript levels compared to the vehicle-treated rats (Table 5). Quantitative PCR was carried out using a PRISM/7700 Sequence Detector with the SYBR Green PCR Core Reagents Kit (Applied Biosystems). Primers were designed to detect rat GLP-1 receptor: 5′-gggatgggctcctctcgta-3′, 5′-cacgcagtattgcatgagca-3′. β-actin (5′-ctgccctggctcctagcac-3′ and 5′-cgctcaggaggagcaatga-3′) was used as the endogenous control. Data from ABI Prism 7700 Sequence Detection System (version 1.7 software) were calibrated to β-actin and the relative quantitation of gene expression was performed using the comparative CT method. Student's t-test was used for statistical analysis.

TABLE 5
Quantitative RTPCR of hippocampal GLP-1
receptor of rats trained in PA.

| Group | Level | P value |
|---|---|---|
| Vehicle vs. Sham | 3.51 | 0.09⁺ |
| | (1.79-6.85) | |
| Gilatide vs. Sham | 0.71 | 0.64 |
| | (0.23-1.92) | |
| Vehicle vs. Gilatide | 4.97 | 0.02* |
| | (1.82-13.5) | |

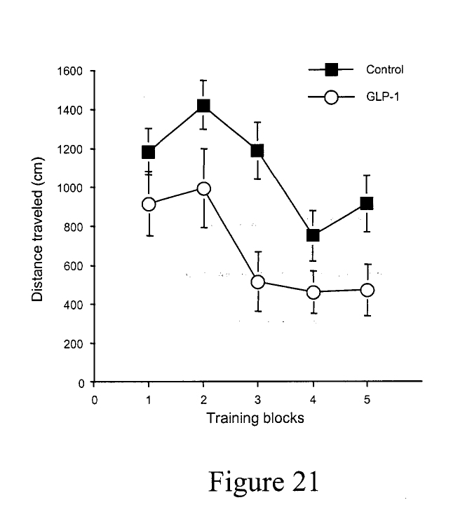

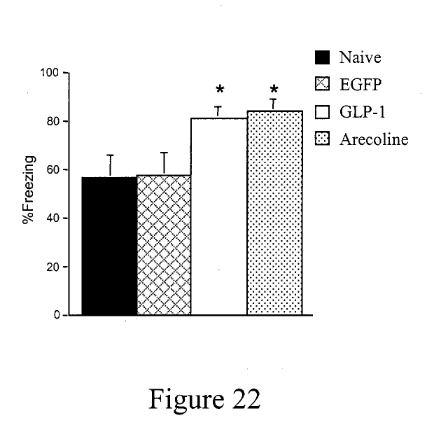

To determine whether increasing GLP-1R levels in the hippocampus would enhance learning, rAAV vectors expressing control EGFP vector and GLP-1R were generated and injected into the hippocampus of rats. See Example 1(vi) for protocol. Three weeks following vector injection, robust hippocampal expression was obtained (data not shown) with transgene mRNA expression in the principal cell groups of the hippocampus. Representative brain sections showed GFP expression in hippocampus, and in situ hybridization for GLP-1R expression in a naïve rat and a rat that received rAAVGLP-1 (data not shown). See Example 1 (vii) for protocol. A separate group of rats treated in the same manner was trained twice daily for 5 days in the MWM. Those rats that overexpressed GLP-1R showed marked enhancement in spatial learning, with reductions in both latency (not shown; Two-way ANOVA: F=25.04(1,80); P<0.001)) and distance traveled (FIG. 21) to locate the hidden platform compared to EGFP controls. The decrease in latency was not due to increased swimming speed (both GLP-1R and EGFP controls were approx. 23 cm/sec) nor stress effects (Table 4). Furthmore, the decreased latency was not due to a disruption in visual acuity and general locomotion and swimming ability, because rats from both groups located a visual platform in a similar manner (approx. 32±3 sec latency). Next, we tested associative learning in rats that over-expressed GLP-1R, using contextual fear conditioning. GLP-1R overexpressing rats showed similar levels of freezing to arecoline-treated animals and significantly greater freezing (FIG. 22) compared to EGFP and naïve control rats.

### Example 10

### Locomotor Activity

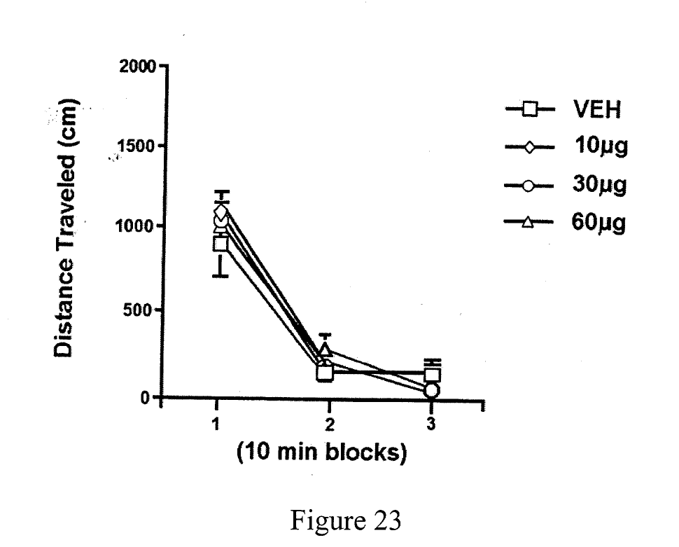

Since drugs that effect arousal and attention generally are psychomotor stimulants, Gilatide was tested in a fully automated and comprehensive locomotor activity apparatus. Rats were pretreated with either 10-60 μg/kg of Gilatide in 5% β cyclodextrin intransally or vehicle (5% β cyclodextrin). Following pretreatment, the rats were placed for 30 minutes in an open field testing chamber (17″×17″×12″ H) where movement was detected every 50 ms by infrared photo beam emitter and detector strips at 1″ and 10″ from the bottom of the chamber. The activity chambers were lined to a PC computer and data was compiled via Activity Monitor Software (4.0, MED Associates, St. Albans, Vt.). The distance traveled did not differ between treatments (□ Vehicle; ⋄ 10 μg; ◯ 30 μg; Δ 60 μg (p>0.05). (FIG. 23)

### Example 11

### Pain Stimulus

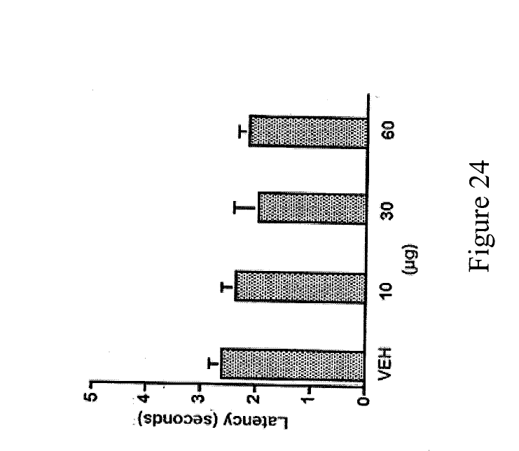

Gilatide administration was further tested in a nonciceptive paradigm. Rats were pretreated with either doses of Gilatide ranging from 10-60 μg/kg (in 5% β cyclodextrin) intransally or vehicle (5% β cyclodextrin). Following treatment, each rat was rolled in a towel with its tail exposed. The tail was then dipped in water maintained at 50±2° C. Latency to remove the tail from the water was measured. Latency measures did not differ between treatments (P>0.05). (FIG. 24)

### Example 12

### Gilatide Increases CREB and MAPK Expression

These studies show that Gilatide peptides and anologs can increase CREB and MAPK expression and can be used as a test of bioactivity of Gilatides.

In one experiment, rats were administered either vehicle, dopamine agonist, or Gilatide 10 μg/kg intranasally. Twenty (20) minutes after treatment the rats were sacrificed and the hippocampus extracted. Samples were then separated into cytosolic and nuclear fractions and probed for CREB and MAPK protein via Western Blot Analysis. (Data not shown) In a second experiment, rats were pretreated with either vehicle or Gilatide 10 μg/kg intranasally and then were either trained in a passive avoidance paradigm, not trained, or sham trained (shock only). The rats were sacrificed two (2) hours after training, and the hippocampus was extracted and processed. The results demonstrated that Gilatide increased CREB protein expression aprroximately 40% in hippocampal nuclear fraction 20 minutes post treatment but not at 2 hour. (Data not shown) Gilatide also increased MAPK protein expression in both cytosolic and nuclear fractions 20 minutes post treatment. (Data not shown)

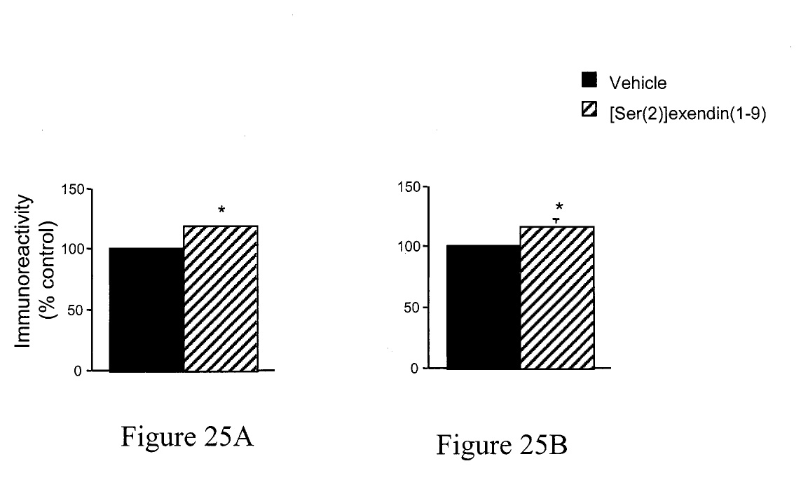

The effect of Gilatide on CREB and MAPK expression in the hippocampus was measured. This study shows the effects of the Gilatide peptide [Ser(2)]exendin(1-9) on MAP kinase. Groups of rats were pretreated with intranasal [Ser(2)]exendin(1-9) or vehicle then sacrificed, and the hippocampus dissected 20 minutes following treatment and probed for MAP kinase activity. Following separation via gel electrophoresis, protein samples (n=6 per group) were transferred to PVDF membrane then probed using MAPK (NEB, MA, 1:200) antibody. Samples were visualized by a chemiluminescent detection system (ECL+; Amersham) with three representative samples shown. Quantitation of immunoreactivity was achieved with NIH Image 1.61. Intranasal administration of Gilatide ([Ser(2)]exendin(1-9)) significantly enhanced MAP kinase immunoreactivity in the cytosolic *P=0.05 (FIGS. 25A), and nuclear *P<0.05 (FIGS. 25B) fractions of hippocampal samples taken following intranasal administration. Gilatide was shown to increase MAP kinase expression in the hippocampus of rats.

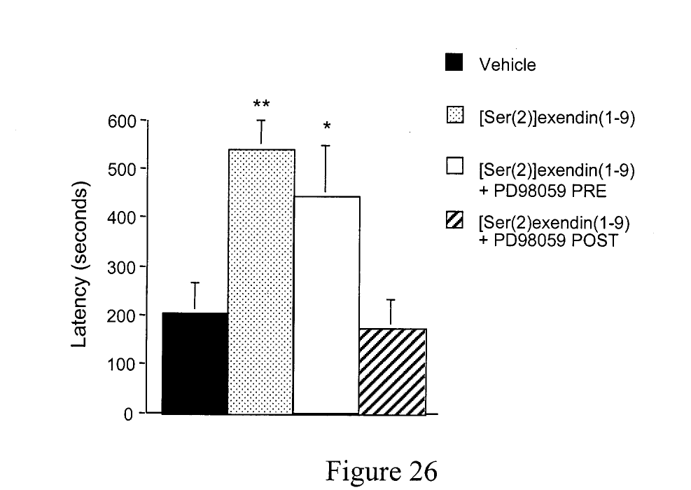

In addition, a study was carried out to determine if the cognitive enhancing effects of the peptide could be blocked by a MAP kinase inhibitor. In addition, the enhancement of associative learning by intranasal [Ser(2)]exendin(1-9) was completely blocked when PD98059 (5 μg, i.c.v.), a specific MEK inhibitor that prevents subsequent ERK/MAPK activation, was administered to rats immediately after training (POST) in the PA paradigm but not when given before (PRE) training (FIG. 26; ▪ vehicle, dotted [Ser(2)]exendin(1-9), □ [Ser(2)]exendin(1-9)+inhibitor PRE, slashed [Ser(2)]exendin(1-9)+inhibitor POST).

### Example 13

### Gilatide and Analogs are Neuroprotective.

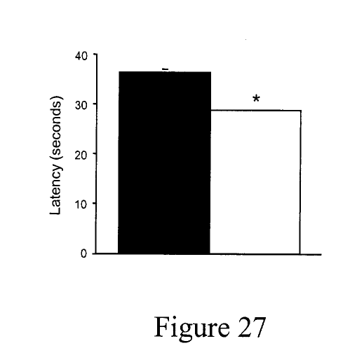

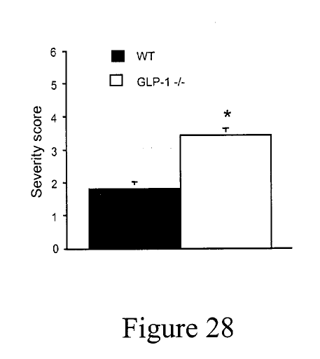

This study shows that Gilatide and analogs have neuroprotective effects when administered to a subject. The effects of the potent neurotoxin, kainic acid (KA), which produces excessive hippocampal excitation and cell loss, particularly in the CA3 subregion when administered systemically, were investigated in GLP-1R+/+ and GLP-1R−/− mice (*P<0.05). Significantly lower seizure latency times were observed in GLP-1R−/− compared to GLP-1R+/+ mice (FIG. 27). Maximal seizure severity scores (B) were greater in GLP-1R−/− compared to GLP-1R+/+ mice (×2, *P<0.03) in response to KA (FIG. 28). Mice were administered KA (20 mg kg −1 i.p.) then placed in a clear container and closely monitored for 40 min. An observer blind to the genotype scored latency to the first clonic-tonic seizure and maximal seizure severity according to protocol previously described (Racine, R. J. Electroencephalogr. Clin. Neurophysiol. 1972, 32: 281): 0, no response; 1, staring; 2, myoclonic jerk; 3, forelimb clonus; 4, rearing; 5, loss of posture-generalized seizure; 6, death. All experiments were filmed and subsequently re-analyzed by an observer blinded to genotype. Mice not demonstrating any signs of seizure were assigned latencies of 40 min. Three days later, mice were sacrificed via intra-cardiac perfusion with 4% formaldehyde and brains processed for Fluorojade staining.

Tissue sections (20 μm) were processed from GLP-1R−/− and GLP-1R+/+ mice 3 days post KA administration. Sections were mounted on slides and left to dry overnight. KA-induced degeneration was determined using anionic fluorescein derivative Fluorojade-B (Histo-Chem Inc., Jefferson, Ark.) (Schmued et al. Brain Res, 2000, 874: 123). Sections were collected every 60 μm for quantitative analysis of hippocampal CA3 degeneration (14 sections per mouse). Fluorojade-positive cells were counted in each hemisphere by a blinded individual and combined to give a total for each GLP-1R−/− and GLP-1R+/+ mouse. Lower cell death was observed in GLP-1R+/+ compared to GLP-1R−/− mice (number of Fluorojade-B positive cells: GLP-1R+/+ 20.66±3.18, GLP-1R−/− 37.00±2.49; P<0.01). Full status epilepticus was observed in only one GLP-1R+/+ mouse compared with six out of ten GLP-1R−/− mice. Immunohistochemical comparison of the CA3 subregion of the hippocampus using Fluorojade B, a fluorochrome stain specific for degenerating neurons (Schmued et al. Brain Res, 2000, 874:123), showed significantly lower cell death in GLP-1R+/+ compared to GLP-1R−/− mice. These results suggest that the GLP-1R may play an important role in neuroprotection.

Parallel experiments determined the effects of [Ser(2)]exendin(1-9) on KA-induced apoptosis in the rat. Rats were administered KA (8 mg kg −1 i.p.) and sacrificed 3 days later. Their brains were processed for TUNEL as described previously (Young et al. Nat. Med. 1999, 5: 448). All sections were scored for TUNEL-positive nuclei by a blinded individual. Sections taken every 150 μm spanning a region between −2.5 mm to −4.6 mm from Bregma were scored, with numbers from both hemispheres collated to give a final mean number for each treatment group. In this study, intranasal [Ser(2)]exendin(1-9) or scrambled peptide was followed 20 minutes later by KA. Three days after insult, the brains were dissected and TdT-mediated dUTP nick end labeling (TUNEL) was used to determine DNA8 degradation in the hippocampus. Compared to the scrambled peptide, [Ser(2)]exendin(1-9) significantly attenuated KA-induced apoptosis in the CA3 region of the hippocampus, as measured by the number of TUNEL-positive cells. Intranasal Gilatide, but not scrambled peptide, decreased the number of TUNEL-positive cells in response to KA. Tunnel-positve cells (apoptotic cells) following KA (8 mg/kg, i.p.) and intranasal administration of either scrambled peptide (the same nine amino acids as Giltide, but in a random order), Gilatide, or nothing (naïve) were 48.43±10.37, 23.00±7.62, and 3.50±1.66, P<0.05), respectively. Thus, Gilatide is shown to have neuroprotective effects.

### Equivalents

Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims. All publications and references are herein expressly incorporated by reference in their entirety.

1. A method for modulating blood glucose in a subject, comprising administering to the subject a therapeutically effective amount of a Gilatide peptide or functional analog thereof that modulates insulin secretion, such that the administration of the Gilatide peptide or functional analog produces an increase in insulin, thereby modulating blood glucose levels.

2. The method of claim 1, wherein the method further comprises the step of modulating a glucose-metabolism disorder in a subject.

3. The method of claim 2, wherein the glucose-metabolism disorder is diabetes.

4. The method of claim 1, wherein the step of administering the therapeutically effective amount of a Gilatide peptide or functional analog is selected from the group comprising intraperitoneal, intracerebroventricular, intradermal, intramuscular, intravenous, subcutaneous, and intranasal delivery.

5. The method of claim 1, wherein the therapeutically effective amount of Gilatide peptide of functional analog is administered intraperitonally.

6. The method of claim 1, wherein the Gilatide peptide or functional analog thereof comprises less than 25 amino acids.

7. The method of claim 1, wherein the Gilatide peptide or functional analog thereof comprises less than 20 amino acids.

8. The method of claim 1, wherein the Gilatide peptide or functional analog thereof comprises less than 15 amino acids.

9. A method of modulating insulin in a subject comprising administering to the subject a pharmaceutical composition comprising a therapeutically effective amount of a Gilatide peptide or functional analog thereof, such that the Gilatide peptide or functional analog thereof interacts with a glucogan-like peptide-1 receptor (GLP-1R), whereby insulin secretion is induced.
